(19) United States
(12) Patent Application Publication
(10) Pub. No.: US 2026/0209686 A1
(43) Pub. Date: Jul. 23, 2026

(54) METHODS AND COMPOSITIONS FOR REFACTORING NITROGEN FIXATION CLUSTERS

(71) Applicant: BIOCONSORTIA, INC., DAVIS, CA (US)

(72) Inventors: THOMAS WILLIAMS, WOODLAND, CA (US); JOHN MALIN, SACRAMENTO, CA (US); KYLE TIPTON, SACRAMENTO, CA (US); DONALD GIBSON, SACRAMENTO, CA (US); DAMIAN CURTIS, DAVIS, CA (US); BETSY ALFORD, DAVIS, CA (US); HONG ZHU, WEST SACRAMENTO, CA (US); COURTNEY REIMCHE, EL DORADO HILLS, CA (US)

(73) Assignee: BIOCONSORTIA, INC., DAVIS, CA (US)

(21) Appl. No.: 18/865,537

(22) PCT Filed: May 17, 2023
(86) PCT No.: PCT/US2023/022584

§ 371 Date: Nov. 13, 2024

Related U.S. Application Data
(60) Provisional application No. 63/343,068, filed on May 17, 2022.

(51) Int. Cl. C12N 1/205; A01N 25/02; A01N 63/25; A01P 21/00; C12N 15/74; C12R 1/01

(52) U.S. Cl. CPC C12N 1/205; A01N 25/02; A01N 63/25; A01P 21/00; C12N 15/74; C12N 2800/101; C12R 2001/01

(57) ABSTRACT

The disclosure relates to genetically modified microorganisms, for example of the Genus Paenibacillus, for the improvement of phenotypes of plants, for example nitrogen availability for plants. Included are novel strains of the microorganisms, microbial consortia, and agricultural compositions comprising the same. Furthermore, the disclosure teaches methods of utilizing the described microorganisms, microbial consortia, and agricultural compositions comprising the same, in methods for imparting beneficial properties to target plant species. In particular aspects, the disclosure provides methods of increasing desirable plant traits in agronomically important species, for example nitrogen fixation, utilization, regulation, uptake, acquisition, tolerance, and/or processing in plants.

### CROSS-REFERENCE TO RELATED APPLICATIONS

This application is a National Stage Entry of International Patent Application No. PCT/US2023/022584 filed on 17 May 2023, which claims the benefit under 35 U.S.C. 119 (e) of U.S. Provisional Patent Application Ser. No. 63/343,068 filed 17 May 2022, herein incorporated by reference in its entirety.

### FIELD

The present disclosure relates to isolated and genetically modified microorganisms that have application, inter alia, in agriculture. The disclosed microorganisms can be utilized in their isolated and biologically pure states, as well as being formulated into agriculturally acceptable compositions. Also disclosed are methods of using the isolated microorganisms or agriculturally acceptable compositions in agricultural applications.

### BACKGROUND

Scientists have estimated that if the global agricultural “yield gap” (which is the difference between the best observed yield and results elsewhere) could be closed, then worldwide crop production would rise by 45-70%. That is, if all farmers, regardless of worldwide location, could achieve the highest attainable yield expected for their respective regions, then a great majority of the deficiencies in worldwide food production could be addressed. However, solving the problem of how to achieve higher yields across a heterogenous worldwide landscape are difficult. Often, yield gaps can be explained by inadequate water, substandard farming practices, inadequate fertilizers, and the non-availability of herbicides and pesticides. However, to vastly increase the worldwide use of water, fertilizers, herbicides, and pesticides, would not only be economically infeasible for most of the world, but would have negative environmental consequences.

Thus, meeting global agricultural yield expectations, by simply scaling up current high-input agricultural systems—utilized in most of the developed world—is simply not feasible.

There is therefore an urgent need in the art for improved methods of increasing crop performance and imparting beneficial traits to desired plant species. The technology described herein include nitrogen fixing bacteria that have been gene edited via refactoring clusters of genes in the nitrogen fixation pathway, to increase the amount of atmospheric nitrogen that is fixed.

### SUMMARY

Included are microorganisms that have application in various fields, including agriculture. The disclosed microorganisms can be utilized in their isolated and biologically pure states, as well as being formulated into agriculturally acceptable compositions. Further provided are agriculturally beneficial microbial consortia, comprising at least two members of the disclosed microorganisms, as well as methods of utilizing said consortia in agricultural applications. In some aspects, genomic modification of the microbes (individual, consortia, and/or communities) are contemplated, for the improvement of microbial traits and the improvement of microbe-associated plants.

The present disclosure addresses this important issue of how to improve plant performance, thereby closing the worldwide yield gap, along with providing ways of imparting other beneficial traits to plant species. The novel genome-edited strains of Paenibacillus described herein improve plant performance by enabling the plant for increased and/or improved nitrogen availability, fixation, uptake, acquisition, tolerance, distribution, regulation, processing, and/or any plurality and/or combination of any of the preceding.

In some embodiments, the plant is non-leguminous crop plant.

In some embodiments, the plant is a dicot. In some embodiments, the plant is a vegetable, herb, ornamental, or fruit plant. In some embodiments, the plant is selected from the group consisting of: kale, spinach, lettuce, carrot, potato, beet, radish, tomato, broccoli, cauliflower, squash, mustard, berry, pepper, greens, pole beans, muskmelon, cucumber, basil, grape, and okra.

In some embodiments, the plant is a monocot. In some embodiments, the plant is a C3 monocot. In some embodiments, the plant is a C4 monocot. In some embodiments, the plant is selected from the group consisting of: maize, wheat, rice, sorghum, sugarcane, onion, bamboo, palm, garlic, ginger, lily, daffodil, iris, orchid, bluebell, tulip, amaryllis, banana, plantain, ginger, turmeric, cardamom, asparagus, pineapple, sedge, rush, leek, forage grass, turf grass, buckwheat, quinoa, chia, and millet.

The solution to increasing crop performance and increasing yield proffered by the present disclosure is not detrimental to the earth's resources, as it does not rely upon increased water consumption or increased input of synthetic chemicals into a system. Rather, the present disclosure utilizes microbes to impart beneficial properties, including increased yields, to desirable plants.

The disclosure therefore offers an environmentally sustainable solution that allows farmers to increase yields of important crops, which is not reliant upon increased utilization of synthetic herbicides and pesticides.

In embodiments, the disclosure provides for an efficient and broadly applicable agricultural platform utilizing microbes and microbial consortia (a plurality of microbes, in some aspects a plurality that improves the health or desired phenotype of the plant, such as an agronomic trait, with which it is associated) that promote one or more desirable plant properties.

The microbes disclosed herein improve the performance of plants, such as crop plants, by both direct and indirect mechanisms. In some aspects, the microbe becomes symbiotic with the plant. In some aspects, the microbe produces a compound (e.g., a metabolite, a toxin, a protein, a lipopeptide, or other composition) that confers a benefit to the plant or that the plant can use for improved characteristics. In some aspects, the microbe improves the solubility of one or more compositions, such as a nutrient, thereby benefitting the plant. In some aspects, the microbe imparts a tolerance to the plant to an exogenous substance such as an herbicide or a pesticide. In some aspects, the microbe produces a composition that is detrimental to a plant pest, such as an insect. In some aspects, the microbe fixes Nitrogen, thereby improving the nutritional status of the plant. Other aspects beyond the exemplary non-limiting aspects listed above are contemplated.

In some embodiments, a single microbe is utilized. In some aspects, the single microbe is isolated and purified. In some aspects, the single microbe is a taxonomic species of bacteria. In some aspects, the single microbe is an identifiable strain of a taxonomic species of bacteria. In some aspects, the single microbe is a novel, newly discovered strain of a taxonomic species of bacteria.

In some aspects, the single microbe—whether a taxonomically identifiable species or strain—is combined with one or more other microbes of a different species or strain. In certain aspects, the combination of two or more microbes forms a consortia or consortium. The terms consortia and consortium are utilized interchangeably.

In certain aspects, the disclosure provides for the development of highly functional microbial consortia that help promote the development and expression of a desired phenotypic or genotypic plant trait. In some embodiments, the consortia of the present disclosure possess functional attributes that are not found in nature, when the individual microbes are living alone. That is, in various embodiments, the combination of particular microbial species into consortia, leads to the microbial combination possessing functional attributes that are not possessed by any one individual member of the consortia when considered alone.

In some embodiments, this functional attribute possessed by the microbial consortia is the ability to impart one or more beneficial properties to a plant species, for example: increased growth, increased yield, increased nutrient utilization (e.g., nitrogen, phosphate, and the like), increased nitrogen utilization efficiency, increased stress tolerance, increased drought tolerance, increased photosynthetic rate, enhanced water use efficiency, increased pathogen resistance, modifications to plant architecture that don't necessarily impact plant yield, but rather address plant functionality, etc. Further contemplated are beneficial properties of pest resistance and/or tolerance, comprising an adverse effect against a nematode, insect, or other pest.

The ability to impart these beneficial properties upon a plant is not possessed, in some embodiments, by the individual microbes as they would occur in nature. Rather, in some embodiments, it is by the hand of man combining these microbes into consortia that a functional composition is developed, said functional composition possessing attributes and functional properties that do not exist in nature. In some embodiments, the consortia may include microbes that have been genetically edited, altered, or modified through the modification of cellular compositions, including DNA, RNA, proteins and/or combinations of the same, via techniques known to those of ordinary skill in the art.

However, in other embodiments, the disclosure provides for individual isolated and biologically pure microbes that are capable of imparting beneficial properties upon a desired plant species, without the need to combine said microbes into consortia.

In some embodiments, the microbe is a strain of the genus Paenibacillus that has been genetically modified to improve nitrogen fixation capabilities. The one or more genetic modifications are characterized as providing improved nitrogen fixation activity to the genetically modified microbe, as compared to a non-genetically modified strain of the microbe.

The disclosure therefore offers an environmentally sustainable solution that allows farmers to increase yields of important crops that is not reliant upon increased utilization of synthetic fertilizer, herbicides, and/or pesticides. For example, in one aspect, the present disclosure describes isolated microbes that are genetically modified to improve the nitrogen fixation ability of the microbe. In some embodiments, an endogenous nif gene of the isolated microbes is genetically modified to improve the nitrogen fixation ability of the microbe.

In some embodiments, the isolated, genetically modified microbes described herein are characterized as having constitutive expression of the nif gene, regardless of the local nitrogen concentration in the environment surrounding the microbe. For examples, in some embodiments, the isolated, genetically modified microbes described herein are characterized as having constitutive expression of the nif gene under nitrogen-limiting conditions. In some embodiments, the isolated, genetically modified microbes described herein are characterized as having constitutive expression of the nif gene under nitrogen-abundant conditions.

Genome modification of Paenibacillus nif gene cluster components can result in improved nitrogen fixation capability for the organism. Approaches include, but are not limited to, the following. Broadly, it is contemplated that genes (nif, anf, vnf) may be added, shuffled, substituted with analogs/homologs, duplicated or other higher-order replicated, introduced from other strains, reordered, and/or any combination of the preceding.

In some aspects, the nifBHDK cluster in a Subgroup I strain is duplicated into that same strain (e.g., nifBHDK-nifBHDK-nifBHDK).

In some aspects, anf and/or vnf may be introduced into Paenibacillus, such as Subgroup I strains (e.g., nifBHDK-anfHDGK-vnfHDGK).

In some aspects, gene expression across nifH, anfH, and/or vnfH may be synchronized in a Subgroup II strain with the same promoter and nifB gene.

In some aspects, the nifB genes in a Subgroup II strain may be streamlined.

In some aspects, different promoters and/or other regulatory elements may be changed, modified, or swapped.

In some aspects, anf and/or vnf gene(s) may be introduced into a strain of Subgroup I, such as Paenibacillus polymyxa. The anf/vnf cluster less sensitive to O2, and introducing into polymyxa may make it less sensitive to oxygen, or function in the absence or limitation of a rare element.

In some aspects, the nifH cluster is modified, for example: duplication of the nitrogen fixation gene cluster using “distant” nitrogen fixation gene cluster or synthetic cluster in a low copy number plasmid-based test system; duplication of the nitrogen fixation gene cluster using Subgroup II cluster with and without orf1 using a plasmid-based test system; duplication of the nitrogen fixation gene cluster using anf cluster using a plasmid-based test system

In some aspects, all or part of the nif cluster is replicated, for example on a plasmid or into the microbe's genome via chromosomal integration.

In some aspects, anf and/or vnf gene(s) may be duplicated.

In some aspects, anf and/or vnf gene(s) may be upregulated.

In some aspects, orf1 may be inserted into the nif cluster of a Subgroup I strain.

In some aspects, the distance between the cluster and the ORI may be altered, to obtain more consistent expression.

In some aspects, duplication of some or all of the cluster within the microbe's genome may be effected, for example by providing multiple copies of nifB, as the instigator of the cluster. The nifB gene of polymyxa is longer than nifBs found in other organisms.

In some aspects, all or part of the nif gene cluster may be transferred from one strain to another, which possesses an improved characteristic (such as colonization of plant tissue).

In some aspects, the nitrogenase protein may be modified to a hybrid of domains of cofactor-utilizing enzymes, to create a novel protein that has more promiscuous activity and not as dependent upon a particular environment as it would be capable of utilizing a variety of cofactors.

In some aspects, the hesA2 gene (found in organisms that also comprise nifH2) may be introduced into P. polymyxa, to enhance the promiscuity of the nitrogenase enzyme.

In some aspects, an artificial dimer of nifH may be created using non-naturally occurring combinations of subunits.

In some aspects, a nifA gene from a near relative of Paenibacillus (e.g., Frankia) is introduced into the Paenibacillus bacterium as a binder for a negative regulator.

In some aspects, the scavenging of 02 activity is upregulated.

In some aspects, the catalase gene is duplicated (or triplicated, or more).

In some aspects, the SOD enzyme is upregulated.

The present disclosure further relates to agricultural compositions that include one or more strains of the isolated, genetically modified microbes disclosed herein and an agriculturally acceptable carrier. In some embodiments, the agricultural compositions include one or more additional agriculturally beneficial agents (e.g. fertilizers, biofertilizers, bionematicides, biostimulants, synthetic pesticides, and/or synthetic herbicides).

Also disclosed herein are methods of imparting one or more beneficial traits to a plant, where the methods include applying an agriculturally effective amount of one or more of the isolated, genetically modified microbes or agricultural compositions disclosed herein.

Any strain disclosed herein may further be combined with one or more additional microbes, which may form a microbial consortia. The microbial consortia can be any combination of one or more individual microbes. In certain embodiments, the microbial consortia comprise two microbes, or three microbes, or four microbes, or five microbes, or six microbes, or seven microbes, or eight microbes, or nine microbes, or 10 microbes, or more than 10 microbes.

Another object of the disclosure is to design a microbial consortium, which is able to perform multidimensional activities in common. In certain aspects, the microbes comprising the consortium act synergistically. In aspects, the effect that the microbial consortium has on a certain plant characteristic is greater than the effect that would be observed had any one individual microbial member of the consortium been utilized singularly. That is, in some aspects, the consortium exhibits a greater than additive effect upon a desired plant characteristic, as compared to the effect that would be found if any individual member of the consortium had been utilized by itself.

In some aspects, the consortia lead to the establishment of other plant-microbe interactions, e.g., by acting as primary colonizers or founding populations that set the trajectory for the future microbiome development.

In embodiments, the disclosure is directed to synergistic combinations (or mixtures) of microbial isolates.

In some aspects, the consortia taught herein provide a wide range of agricultural applications, including: improvements in yield of grain, fruit, and flowers; improvements in growth of plant parts; improved ability to utilize nutrients (e.g., nitrogen, phosphate, and the like), improved resistance to disease; biopesticidal effects including improved resistance to fungi, insects, and nematodes; improved survivability in extreme climate; and improvements in other desired plant phenotypic characteristics. Significantly, these benefits to plants and/or adverse effect on targeted pests and/or pathogens can be obtained without any hazardous side effects to the environment.

In some aspects, the individual microbes of the disclosure, or consortia comprising same, can be combined into an agriculturally acceptable composition.

In some embodiments, the agricultural compositions of the present disclosure include, but are not limited to: wetters, compatibilizing agents, antifoam agents, cleaning agents, sequestering agents, drift reduction agents, neutralizing agents, buffers, corrosion inhibitors, dyes, odorants, spreading agents, penetration aids, sticking agents, binders, dispersing agents, thickening agents, stabilizers, emulsifiers, freezing point depressants, antimicrobial agents, fertilizers, pesticides, nematicides, insecticides, herbicides, inert carriers, polymers, and the like.

In one embodiment of the present disclosure, the microbes (including isolated single species, or strains, consortia, or compositions thereof, such as metabolites), are supplied in the form of seed coatings or other applications to the seed. In embodiments, the seed coating may be applied to a naked and untreated seed. In other embodiments, the seed coating may be applied to a previously treated seed. Thus, in some embodiments, the present disclosure teaches a method of treating a seed comprising applying an isolated bacterial strain or a microbial consortium to a seed. In certain embodiments, the isolated bacterial strain or microbial consortium is applied as an agricultural composition including an agriculturally acceptable carrier. In some embodiments, the agricultural compositions may be formulated as: a soil drench, a foliar spray, a dip treatment, an in-furrow treatment, a soil amendment, granules, a broadcast treatment, a post-harvest disease control treatment, or a seed treatment. In some embodiments, the agricultural compositions may be applied alone in or in rotation spray programs with other agricultural products. In some embodiments, the agricultural compositions may be compatible with tank mixing. In some embodiments, the agricultural compositions may be compatible with tank mixing with other agricultural products. In some embodiments, the agricultural compositions may be compatible with equipment used for ground, aerial, and irrigation applications.

In some embodiments, the applied microbes may become endophytic and consequently may be present in the growing plant that was treated and its subsequent offspring. In other embodiments the microbes might be applied at the same time as a co-treatment with seed treatments.

In one embodiment of the present disclosure, the microbes are supplied in the form of granules, or plug, or soil drench that is applied to the plant growth media. In other embodiments, the microbes are supplied in the form of a foliar application, such as a foliar spray or liquid composition. The foliar spray or liquid application may be applied to a growing plant or to a growth media, e.g., soil.

In other embodiments, the microbes (including isolated single species, or strains, or consortia, or compositions thereof, such as metabolites) are supplied as fertilizers, pesticides, or other amendments that may be applied to soil. In some embodiments, the microbes are supplied as fertilizers, pesticides, or other amendments that are applied to soil prior to planting. In some embodiments, the microbes are supplied as fertilizers, pesticides, or other amendments that are applied to soil concurrent with planting. In some embodiments, the microbes are supplied as fertilizers, pesticides, or other amendments that are applied to soil after planting.

In other embodiments of the present disclosure, the microbes (including isolated single species or strains, or consortia) and/or compositions thereof (e.g., metabolites) are supplied in the form of a post-harvest disease control application.

In embodiments, the agricultural compositions of the disclosure can be formulated as: (1) solutions; (2) wettable powders; (3) dusting powders; (4) soluble powders; (5) emulsions or suspension concentrates; (6) seed dressings, (7) tablets; (8) water-dispersible granules; (9) water soluble granules (slow or fast release); (10) microencapsulated granules or suspensions; (11) as irrigation components, and (12) a component of fertilizers, pesticides, and other compatible amendments, among others. In certain aspects, the compositions may be diluted in an aqueous medium prior to conventional spray application. The compositions of the present disclosure can be applied to the soil, plant, seed, rhizosphere, rhizosheath, or other area to which it would be beneficial to apply the microbial compositions.

Still another object of the disclosure relates to the agricultural compositions being formulated to provide a high colony forming units (CFU) bacterial population or consortia. In some aspects, the agricultural compositions have adjuvants that provide for a pertinent shelf life. In embodiments, the CFU concentration of the taught agricultural compositions is higher than the concentration at which the microbes would exist naturally, outside of the disclosed methods. In another embodiment, the agricultural composition contains the microbial cells in a concentration of 10\hat{ }2-10\hat{ }12 CFU per gram of the carrier or 10\hat{ }5-10\hat{ }9 CFU per gram of the carrier. In an aspect, the microbial cells are applied as a seed coat directly to a seed at a concentration of 10\hat{ }5-10\hat{ }9 CFU. In other aspects, the microbial cells are applied as a seed overcoat on top of another seed coat at a concentration of 10\hat{ }5-10\hat{ }9 CFU. In other aspects, the microbial cells are applied as a co-treatment together with another seed treatment at a rate of 10\hat{ }5-10\hat{ }9 CFU.

In aspects, the disclosure is directed to agricultural microbial formulations that promote plant growth. In aspects, the disclosure provides for the taught isolated microbes, and consortia comprising same, to be formulated as an agricultural bioinoculant. The taught bioinoculants can be applied to plants, seeds, or soil, or combined with fertilizers, pesticides, and other compatible amendments. Suitable examples of formulating bioinoculants comprising isolated microbes can be found in U.S. Pat. No. 7,097,830, which is herein incorporated by reference.

The disclosed microbial formulations can: lower the need for nitrogen containing fertilizers, solubilize minerals, provide biopesticidal protection of the plants, protect plants against pathogens (e.g., fungi, insects, and nematodes), and make available to the plant valuable nutrients, such as nitrogen and/or phosphate, thus reducing and eliminating the need for using chemical pesticides and chemical fertilizers.

In some embodiments, the isolated and biologically pure microbes of the present disclosure can be utilized, in a method of imparting one or more beneficial properties or traits to a desired plant species.

In some embodiments, the agriculturally acceptable composition containing isolated and biologically pure microbes of the present disclosure can be utilized, in a method of imparting one or more beneficial properties or traits to a desired plant species.

In some embodiments, the consortia of the present disclosure can be utilized, in a method of imparting one or more beneficial properties or traits to a desired plant species.

In some embodiments, the agriculturally acceptable composition containing consortia of the present disclosure can be utilized, in a method of imparting one or more beneficial properties or traits to a desired plant species.

The present disclosure provides that a plant element or plant part can be effectively augmented, by coating said plant element or plant part with an isolated microbe or microbial consortia, in an amount that is not normally found on the plant element or plant part.

Some embodiments described herein are methods for preparing an agricultural seed composition, or seed coating, comprising: contacting the surface of a seed with a formulation comprising a purified microbial population that comprises at least one isolated microbe that is heterologous to, or rarely present on the seed. Further embodiments entail preparing an agricultural plant composition, comprising: contacting the surface of a plant with a formulation comprising a purified microbial population that comprises at least one isolated microbe that is heterologous to the plant. In other aspects, the formulation or microbe(s) is (are) introduced into the interior of the seed, for example into the cotyledon or the embryo other seed tissue.

In some aspects, applying an isolated microbe, microbial consortia, exudate, metabolite, and/or agricultural composition of the disclosure to a seed or plant modulates a trait of agronomic importance. The trait of agronomic importance can be, e.g., disease resistance, drought tolerance, heat tolerance, cold tolerance, salinity tolerance, metal tolerance, herbicide tolerance, chemical tolerance, improved water use efficiency, improved nitrogen utilization, improved resistance to nitrogen stress, improved nitrogen fixation, improved nutrient utilization (e.g., phosphate, potassium, and the like), pest resistance, herbivore resistance, pathogen resistance, reduced pathogen levels (e.g., via the excretion of metabolites that impair pathogen survival), increased yield, increased yield under water limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, faster seed germination, altered seed carbohydrate composition, altered seed oil composition, number of pods, delayed senescence, stay-green, and altered seed protein composition. In some aspects, at least 2, 3, 4, or more traits of agronomic importance are modulated. In some aspects, the modulation is a positive effect on one of the aforementioned agronomic traits.

In some aspects, the isolated microbes, consortia, and/or agricultural compositions of the disclosure can be applied to a plant, in order to modulate or alter a plant characteristic such as altered oil content, altered protein content, altered seed carbohydrate composition, altered seed oil composition, altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, decreased biomass, increased root length, decreased root length, increased seed weight, increased shoot length, decreased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome relative to a reference plant.

In some embodiments, the agricultural formulations taught herein comprise at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient.

The methods described herein can include contacting a seed or plant with at least 100 CFU or spores, at least 300 CFU or spores, at least 1,000 CFU or spores, at least 3,000 CFU or spores, at least 10,000 CFU or spores, at least 30,000 CFU or spores, at least 100,000 CFU or spores, at least 300,000 CFU or spores, at least 1,000,000 CFU or spores or more, of the microbes taught herein.

The methods described herein can include contacting a seed or plant with a composition that includes metabolites produced by a single microbe or microbial consortium disclosed herein. In some aspects, the methods include contacting a seed or plant with a composition that includes at least 1 mg of metabolites produced by a single microbe or microbial consortium disclosed herein. In some aspects, the methods include contacting a seed or plant with a composition that includes at least 10 mg of metabolites produced by a single microbe or microbial consortium disclosed herein. In some aspects, the methods include contacting a seed or plant with a composition that includes at least 100 mg of metabolites produced by a single microbe or microbial consortium disclosed herein. In some aspects, the methods include contacting a seed or plant with a composition that includes at least 1 g of metabolites produced by a single microbe or microbial consortium disclosed herein. In some aspects, the methods include contacting a seed or plant with a composition that includes at least 10 g of metabolites produced by a single microbe or microbial consortium disclosed herein. In some aspects, the methods include contacting a seed or plant with a composition that includes at least 100 g of metabolites produced by a single microbe or microbial consortium disclosed herein. In some aspects, the methods include contacting a seed or plant with a composition that includes at least 1 kg of metabolites produced by a single microbe or microbial consortium disclosed herein. In some aspects, the methods include contacting a seed or plant with a composition that includes greater than 1 kg of metabolites produced by a single microbe or microbial consortium disclosed herein.

In some embodiments of the methods described herein, an isolated microbe of the disclosure is present in a formulation in an amount effective to be detectable within and/or on a target tissue of an agricultural plant. For example, the microbe is detected in an amount of at least 100 CFU or spores, at least 300 CFU or spores, at least 1,000 CFU or spores, at least 3,000 CFU or spores, at least 10,000 CFU or spores, at least 30,000 CFU or spores, at least 100,000 CFU or spores, at least 300,000 CFU or spores, at least 1,000,000 CFU or spores, or more, in and/or on a target tissue of a plant. Alternatively or in addition, the microbes of the disclosure may be present in a formulation in an amount effective to increase the biomass and/or yield of a plant that has had such a formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with a reference agricultural plant that has not had the formulations of the disclosure applied. Alternatively or in addition, the microbes of the disclosure may be present in a formulation in an amount effective to detectably modulate an agronomic trait of interest of a plant that has had such a formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with a reference agricultural plant that has not had the formulations of the disclosure applied.

In some embodiments of the methods described herein, one or more metabolites isolated from the microbes or consortia of the disclosure are present in a formulation in an amount effective to be detectable within and/or on a target tissue of an agricultural plant. For example, the metabolites are detected in an amount of at least 1 mg, at least 10 mg, at least 50 mg, at least 100 mg, at least 200 mg, at least 400 mg, at least 600 mg, at least 800 mg, at least 1 g, or more, in and/or on a target tissue of a plant. Alternatively or in addition, the metabolites isolated from the microbes and consortia of the disclosure may be present in a formulation in an amount effective to increase the biomass and/or yield of a plant that has had such a formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with a reference agricultural plant that has not had the formulations of the disclosure applied. Alternatively or in addition, the metabolites isolated from the microbes and consortia of the disclosure may be present in a formulation in an amount effective to detectably modulate an agronomic trait of interest of a plant that has had such a formulation applied thereto, by at least 1%, at least 2%, at least 3%, at least 5%, at least 10%, at least 15%, at least 20%, at least 30%, at least 40%, at least 50%, at least 60%, at least 70%, at least 80%, at least 90%, at least 100%, or more, when compared with a reference agricultural plant that has not had the formulations of the disclosure applied.

In some embodiments, the agricultural compositions taught herein are shelf-stable. In some aspects, the microbes taught herein are freeze-dried. In some aspects, the microbes taught herein are spray-dried. In some aspects, the microbes taught herein are placed in a liquid formulation. In some aspects, the microbes taught herein are present on granules,

Also described herein are a plurality of isolated microbes confined within an object selected from the group consisting of: bottle, jar, ampule, package, vessel, bag, box, bin, envelope, carton, container, silo, shipping container, truck bed, and case.

In some aspects, combining a selected plant species with a disclosed microbe—operational taxonomic unit (OTU), strain, or composition comprising any of the aforementioned—leads to improved yield from crops and generation of products thereof. Therefore, in one aspect, the present disclosure provides a synthetic combination of a seed of a first plant and a preparation of a microbe(s) that is coated onto the surface of the seed of the first plant, such that the microbe is present at a higher level on the surface of the seed, than is present on the surface of an uncoated reference seed. In another aspect, the present disclosure provides a synthetic combination of a part of a first plant and a preparation of a microbe(s) that is coated onto the surface of the part of the first plant, such that the microbe is present at a higher level on the surface of the part of the first plant, than is present on the surface of an uncoated reference plant part. The aforementioned methods can be used alone, or in parallel with plant breeding and transgenic technologies.

In some embodiments, the Paenibacillus strain is described in Table 1. In some embodiments, the Paenibacillus strain comprises a polynucleotide sequence sharing at least 90% identity with any one or more of SEQID NOs. 1-52. In some embodiments, the Paenibacillus strain is a species selected from the group consisting of: polymyxa, tritici, albidus, anaericanus, azotifigens, borealis, donghaensis, ehimensis, graminis, jilunlii, odorifer, panacisoli, phoenicis, pocheonensis, rhizoplanae, silage, taohuashanense, thermophilus, typhae, and wynnii. In some embodiments, the Paenibacillus strain is of Subgroup I. In some embodiments, the Paenibacillus strain is of Subgroup II.

In some embodiments, the isolated bacterial strain has substantially similar morphological and physiological characteristics as an isolated bacterial strain of the present disclosure. In some embodiments, the isolated bacterial strain has substantially similar genetic characteristics as an isolated bacterial strain of the present disclosure. In some embodiments, the isolated bacterial strain is a mutant, naturally occurring or man-made, of an isolated bacterial strain of the present disclosure. In some embodiments, the isolated bacterial strain is a genetically edited, altered, or modified bacterial strain. In some embodiments, an isolated bacterial strain of the present disclosure is in substantially pure culture. In some embodiments, an isolated bacterial strain of the present disclosure is in pure culture. In some embodiments, an isolated bacterial strain of the present disclosure is in a cell fraction, extract or supernatant.

In some embodiments, progeny and/or mutants of an isolated bacterial strain of the present disclosure are contemplated. In some embodiments, progeny, mutants, and/or genetically modified versions of an isolated bacterial strain of the present disclosure are contemplated.

In some embodiments, a cell-free or inactivated preparation of an isolated bacterial strain of the present disclosure is contemplated, or a mutant of said isolated bacterial strain. In some embodiments, a cell-free or inactivated preparation of an isolated bacterial strain of the present disclosure is contemplated, or a mutant or genetically edited, altered, or modified variant of said isolated bacterial strain. In some embodiments, a metabolite produced by an isolated bacterial strain of the present disclosure is contemplated, or a mutant of said isolated bacterial strain. In some embodiments, a metabolite produced by an isolated bacterial strain of the present disclosure is contemplated, or a mutant or genetically modified variant of said isolated bacterial strain.

In some embodiments, an agricultural composition comprises an isolated bacterial strain and an agriculturally acceptable carrier. The isolated bacterial strain may be present in the composition at 1×10\hat{ }2 to 1×10\hat{ }12 CFU per gram. The agricultural composition may be formulated as a seed coating.

In some embodiments, a method of imparting at least one beneficial trait upon a plant species comprises applying an isolated bacterial strain to the plant or to a growth medium in which said plant is located. In some embodiments, a method of imparting at least one beneficial trait upon a plant species comprises applying an agricultural composition of the present disclosure to the plant or to a growth medium in which the plant is located.

In some embodiments, the plant is non-leguminous crop plant. In some embodiments, the plant is a monocot. In some embodiments, the plant is a C3 monocot. In some embodiments, the plant is a C4 monocot. In some embodiments, the plant is selected from the group consisting of: maize, wheat, rice, sorghum, sugarcane, onion, bamboo, palm, garlic, ginger, lily, daffodil, iris, orchid, bluebell, tulip, amaryllis, banana, plantain, ginger, turmeric, cardamom, asparagus, pineapple, sedge, rush, leek, forage grass, buckwheat, quinoa, chia, and millet.

In some embodiments, the present disclosure teaches a method of growing a plant having at least one beneficial trait. In some embodiments, the method comprises applying an isolated bacterial strain or microbial consortium to the seed of a plant; sowing or planting the seed; and growing the plant. In certain embodiments, the isolated bacterial strain or microbial consortium is applied as an agricultural composition that further includes an agriculturally acceptable carrier.

In some embodiments, the microbial consortium has substantially similar morphological and physiological characteristics as a microbial consortium of the present disclosure. In some embodiments, the microbial consortium has substantially similar genetic characteristics as a microbial consortium of the present disclosure. In some embodiments, the microbial consortium is in substantially pure culture. In some embodiments, a subsequent generation of any microbe of the microbial consortium is contemplated. In some embodiments, a mutant of any microbe of the microbial consortium is contemplated. In some embodiments, a genetically edited, altered, or modified variant of any microbe of the microbial consortium is contemplated. In some embodiments, a cell-free or inactivated preparation of the microbial consortium, or a mutant or genetically edited, altered, or modified variant of any microbe in the microbial consortium, is contemplated. In some embodiments, a metabolite produced by the microbial consortium, or a mutant or genetically edited, altered, or modified variant of any microbe in the microbial consortium, is contemplated.

In some embodiments, an agricultural composition comprises a microbial consortium and an agriculturally acceptable carrier. The microbial consortium of the agricultural composition may be present in the composition at 1×10\hat{ }3 to 1×10\hat{ }12 bacterial cells per gram. In some embodiments, the agricultural composition is formulated as a seed coating. In some embodiments, a method of imparting at least one beneficial trait upon a plant species comprises applying a microbial consortium to said plant, or to a growth medium in which said plant is located. In some embodiments, a method of imparting at least one beneficial trait upon a plant species, comprising applying the agricultural composition to the plant, or to a growth medium in which said plant is located.

### DETAILED DESCRIPTION

While the following terms are believed to be well understood by one of ordinary skill in the art, the following are set forth to facilitate explanation of the presently disclosed subject matter.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “microorganism” or “microbe” should be taken broadly. These terms are used interchangeably and include, but are not limited to, the two prokaryotic domains, Bacteria and Archaea, as well as eukaryotic Fungi and Protists. In some embodiments, the disclosure refers to the “microbes” of Table 1, or the “microbes” of various other tables or paragraphs present in the disclosure. This characterization can refer to not only the identified taxonomic bacterial genera of the tables, but also the identified taxonomic species, as well as the various novel and newly identified bacterial strains of said tables.

As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms) or more than one cell (e.g., multi-cellular microorganism). A “population of microorganisms” may thus refer to a multiple cells of a single microorganism, in which the cells share common genetic derivation.

As used herein, the term “bacterium” or “bacteria” refers in general to any prokaryotic organism, and may reference an organism from either Kingdom Eubacteria (Bacteria), Kingdom Archaebacteria (Archaea), or both. In some cases, bacterial genera or other taxonomic classifications may be in taxonomic flux, have been reassigned due to various reasons (such as but not limited to the evolving field of whole genome sequencing), and/or may be variable based on methodology, and it is understood that such nomenclature variabilities are within the scope of any claimed taxonomy. For example, certain species of the genus Erwinia have been described in the literature as belonging to genus Pantoea (Zhang, Y., Qiu, S. Examining phylogenetic relationships of Erwinia and Pantoea species using whole genome sequence data. Antonie van Leeuwenhoek 108, 1037-1046 (2015).).

The term “16S” refers to the DNA sequence of the 16S ribosomal RNA (rRNA) sequence of a bacterium. 16S rRNA gene sequencing is a well-established method for studying phylogeny and taxonomy of bacteria. As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, J. W. One Fungus=One Name: DNA and fungal nomenclature twenty years after PCR. IMA Fungus 2, 113-120 (2011).). With the development of genomic sequencing, it became evident that taxonomic classification based on molecular phylogenetics did not align with morphological-based nomenclature (Shenoy, B. D., Jeewon, R. and Hyde, K. D. (2007). Impact of DNA sequence-data on the taxonomy of anamorphic fungi. Fungal Diversity 26:1-54.). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, D. L. Managing and coping with names of pleomorphic fungi in a period of transition. IMA Fungus 3, 15-24 (2012)).

The term “Internal Transcribed Spacer” (“ITS”) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (“LSU”) sequence is used to identify fungi. LSU gene sequencing is a well-established method for studying phylogeny and taxonomy of fungi. Some fungal microbes of the present invention may be described by an ITS sequence and some may be described by an LSU sequence. Both are understood to be equally descriptive and accurate for determining taxonomy.

The term “microbial consortia” or “microbial consortium” refers to a subset of a microbial community of individual microbial species, or strains of a species, which can be described as carrying out a common function, or can be described as participating in, or leading to, or correlating with, a recognizable parameter or plant phenotypic trait. The community may comprise one or more species, or strains of a species, of microbes. In some instances, the microbes coexist within the community symbiotically.

The term “microbial community” means a group of microbes comprising two or more species or strains. Unlike microbial consortia, a microbial community does not have to be carrying out a common function, or does not have to be participating in, or leading to, or correlating with, a recognizable parameter or plant phenotypic trait.

The term “accelerated microbial selection” or “AMS” is used interchangeably with the term “directed microbial selection” or “DMS” and refers to the iterative selection methodology that was utilized, in some embodiments of the disclosure, to derive the claimed microbial species or consortia of said species.

As used herein, “isolate,” “isolated,” “isolated microbe,” and like terms, are intended to mean that the one or more microorganisms has been separated from at least one of the materials with which it is associated in a particular environment (for example soil, water, plant tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an agricultural carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g., In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), affirmed in part, reversed in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

With respect to microbes, the term “modified” means that the microbe has been changed in some way, as compared to the natural state in which it was found. In this context, “modified” is synonymous with “engineered”, and indicates that the hand of man was involved with creating the modification. In some cases, the modification includes the change of a polynucleotide within the microbe, for example in its genome. Modifications may include deletion, insertion, replacement, and/or chemical alteration of at least one nucleotide, and may result in a change in the phenotype of the microbe (e.g., upregulation of a particular pathway, downregulation of a particular pathway, knockout of a gene or protein function) and/or a change in the phenotype of another, heterologous organism with which the microbe is or becomes associated.

The term “growth medium” as used herein, is any medium which is suitable to support growth of a plant. By way of example, the media may be natural or artificial including, but not limited to: soil, potting mixes, bark, vermiculite, hydroponic solutions alone and applied to solid plant support systems, and tissue culture gels. It should be appreciated that the media may be used alone or in combination with one or more other media. It may also be used with or without the addition of exogenous nutrients and physical support systems for roots and foliage.

In one embodiment, the growth medium is a naturally occurring medium such as soil, sand, mud, clay, humus, regolith, rock, or water. In another embodiment, the growth medium is artificial. Such an artificial growth medium may be constructed to mimic the conditions of a naturally occurring medium; however, this is not necessary. Artificial growth media can be made from one or more of any number and combination of materials including sand, minerals, glass, rock, water, metals, salts, nutrients, water. In one embodiment, the growth medium is sterile. In another embodiment, the growth medium is not sterile.

The medium may be amended or enriched with additional compounds or components, for example, a component which may assist in the interaction and/or selection of specific groups of microorganisms with the plant and each other. For example, antibiotics (such as penicillin) or sterilants (for example, quaternary ammonium salts and oxidizing agents) could be present and/or the physical conditions (such as salinity, plant nutrients (for example organic and inorganic minerals (such as phosphorus, nitrogenous salts, ammonia, potassium and micronutrients such as cobalt and magnesium), pH, and/or temperature) could be amended.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. As used herein, the term “plant element” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Progeny, variants, and mutants of the regenerated plants are also included within the scope of the invention, provided that these parts comprise the introduced polynucleotides.

A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant part” is synonymous to a “portion” of a plant, and refers to any part of the plant, and can include distinct tissues and/or organs, and may be used interchangeably with the term “tissue” throughout.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of an organism, produced via sexual or asexual reproduction.

“Grain” is intended to mean the mature seed produced by commercial growers for purposes other than growing or reproducing the species.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, the term “cultivar” refers to a variety, strain, or race, of plant that has been produced by horticultural or agronomic techniques and is not normally found in wild populations.

As used herein, “improved” should be taken broadly to encompass improvement of a characteristic of a plant, as compared to a control plant, or as compared to a known average quantity associated with the characteristic in question. For example, “improved” plant biomass associated with application of a beneficial microbe, or consortia, of the disclosure can be demonstrated by comparing the biomass of a plant treated by the microbes taught herein to the biomass of a control plant not treated. Alternatively, one could compare the biomass of a plant treated by the microbes taught herein to the average biomass normally attained by the given plant, as represented in scientific or agricultural publications known to those of skill in the art. In the present disclosure, “improved” does not necessarily demand that the data be statistically significant (e.g., p<0.05); rather, any quantifiable difference demonstrating that one value (e.g., the average treatment value) is different from another (e.g., the average control value) can rise to the level of “improved.”

As used herein, “inhibiting and suppressing” and like terms should not be construed to require complete inhibition or suppression, although this may be desired in some embodiments.

As used herein, the term “genotype” refers to the genetic makeup of an individual cell, cell culture, tissue, organism (e.g., a plant), or group of organisms.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein

“Agronomic trait potential” is intended to mean a capability of a plant element for exhibiting a phenotype, preferably an improved agronomic trait, at some point during its life cycle, or conveying said phenotype to another plant element with which it is associated in the same plant.

As used herein, the term “molecular marker”, “marker”, or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed by the average person skilled in molecular-biological techniques.

As used herein, the term “trait” refers to a characteristic or phenotype. For example, in the context of some embodiments of the present disclosure, yield of a crop relates to the amount of marketable biomass produced by a plant (e.g., fruit, fiber, grain). Desirable traits may also include other plant characteristics, including but not limited to: water use efficiency, nutrient use efficiency, production, mechanical harvestability, fruit maturity, shelf life, pest/disease resistance, early plant maturity, tolerance to stresses, etc. A trait may be inherited in a dominant or recessive manner, or in a partial or incomplete-dominant manner. A trait may be monogenic (i.e., determined by a single locus) or polygenic (i.e., determined by more than one locus) or may also result from the interaction of one or more genes with the environment.

As used herein, the term “phenotype” refers to the observable characteristics of an individual cell, cell culture, organism (e.g., a plant), or group of organisms which results from the interaction between that individual's genetic makeup (i.e., genotype) and the environment.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue”, “homolog”, or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are Mac Vector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

As used herein, the term “nucleotide change” refers to, e.g., nucleotide substitution, deletion, insertion, chemical alteration, or any of the preceding, as is well understood in the art.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full-length molecule, up to and including the full-length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g., at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g., Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

In some embodiments, the cell or organism has at least one heterologous trait. As used herein, the term “heterologous trait” refers to a phenotype imparted to a cell or organism by an exogenous molecule or other organism (e.g., a microbe), DNA segment, heterologous polynucleotide or heterologous nucleic acid.

Various changes in phenotype are of interest to the present disclosure, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present disclosure

A “synthetic combination” can include a combination of a plant and a microbe of the disclosure. The combination may be achieved, for example, by coating the surface of a seed of a plant, such as an agricultural plant, or host plant tissue (root, stem, leaf, etc.), with a microbe of the disclosure. Further, a “synthetic combination” can include a combination of microbes of various strains or species. Synthetic combinations have at least one variable that distinguishes the combination from any combination that occurs in nature. That variable may be, inter alia, a concentration of microbe on a seed or plant tissue that does not occur naturally, or a combination of microbe and plant that does not naturally occur, or a combination of microbes or strains that do not occur naturally together. In each of these instances, the synthetic combination demonstrates the hand of man and possesses structural and/or functional attributes that are not present when the individual elements of the combination are considered in isolation.

In some embodiments, a microbe can be “endogenous” to a seed or plant. As used herein, a microbe is considered “endogenous” to a plant or seed, if the microbe is derived from the plant specimen from which it is sourced. That is, if the microbe is naturally found associated with said plant. In embodiments in which an endogenous microbe is applied to a plant, then the endogenous microbe is applied in an amount that differs from the levels found on the plant in nature. Thus, a microbe that is endogenous to a given plant can still form a synthetic combination with the plant, if the microbe is present on said plant at a level that does not occur naturally.

In some embodiments, a composition (such as a microbe) can be “heterologous” (also termed “exogenous”) to another composition (such as a seed or plant), and in some aspects is referred to herein as a “heterologous composition”. As used herein, a microbe is considered “heterologous” to a plant or seed, if the microbe is not derived from the plant specimen from which it is sourced. That is, if the microbe is not naturally found associated with said plant. For example, a microbe that is normally associated with leaf tissue of a maize plant is considered exogenous to a leaf tissue of another maize plant that naturally lacks said microbe. In another example, a microbe that is normally associated with a maize plant is considered exogenous to a wheat plant that naturally lacks said microbe.

A composition is “heterologously disposed” when mechanically or manually applied, artificially inoculated, associated with, or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment (for example, drought). In some embodiments, such a manner is contemplated to be selected from the group consisting of: the presence of the microbe; presence of the microbe in a different number of cells, concentration, or amount; the presence of the microbe in a different plant element, tissue, cell type, or other physical location in or on the plant; the presence of the microbe at different time period, e.g., developmental phase of the plant or plant element, time of day, time of season, and combinations thereof. In some embodiments, “heterologously disposed” means that the microbe being applied to a different tissue or cell type of the plant element than that in which the microbe is naturally found. In some embodiments, “heterologously disposed” means that the microbe is applied to a developmental stage of the plant element, seedling, or plant in which said microbe is not naturally associated, but may be associated at other stages. For example, if a microbe is normally found at the flowering stage of a plant and no other stage, a microbe applied at the seedling stage may be considered to be heterologously disposed. In some embodiments, a microbe is heterologously disposed the microbe is normally found in the root tissue of a plant element but not in the leaf tissue, and the microbe is applied to the leaf. In another non-limiting example, if a microbe is naturally found in the mesophyll layer of leaf tissue but is being applied to the epithelial layer, the microbe would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the microbe being applied is at a greater concentration, number, or amount of the plant element, seedling, or plant, than that which is naturally found in said plant element, seedling, or plant. For example, a microbe is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said microbe. In another non-limiting example, a microbe that is naturally found in a tissue of a cupressaceous tree would be considered heterologous to tissue of a maize, wheat, cotton, soybean plant. In another example, a microbe that is naturally found in leaf tissue of a maize, spring wheat, cotton, soybean plant is considered heterologous to a leaf tissue of another maize, spring wheat, cotton, soybean plant that naturally lacks said microbe, or comprises the microbe in a different quantity.

Microbes can also be “heterologously disposed” on a given plant tissue. This means that the microbe is placed upon a plant tissue that it is not naturally found upon. For instance, if a given microbe only naturally occurs on the roots of a given plant, then that microbe could be exogenously applied to the above-ground tissue of a plant and would thereby be “heterologously disposed” upon said plant tissue. As such, a microbe is deemed heterologously disposed, when applied on a plant that does not naturally have the microbe present or does not naturally have the microbe present in the number that is being applied.

The compositions and methods herein may provide for a “modulated” “agronomic trait” or “trait of agronomic importance” to a host plant, which may include, but not be limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation. By the term “modulated”, it is intended to refer to a change in an agronomic trait that is changed by virtue of the presence of the microbe(s), exudate, broth, metabolite, etc. In aspects, the modulation provides for the imparting of a beneficial trait.

### Microbes and Microorganisms

As used herein the term “microorganism” should be taken broadly. It includes, but is not limited to, prokaryotic Bacteria and Archaea, as well as eukaryotic Fungi and Protists.

In a particular embodiment, the microorganism is an endophyte, or an epiphyte, or a microorganism inhabiting the plant rhizosphere or rhizosheath. That is, the microorganism may be found present in the soil material adhered to the roots of a plant or in the area immediately adjacent a plant's roots.

In one embodiment, the microorganism is an endophyte. Endophytes may benefit host plants by preventing pathogenic organisms from colonizing them. Extensive colonization of the plant tissue by endophytes creates a “barrier effect,” where the local endophytes outcompete and prevent pathogenic organisms from taking hold. Endophytes may also produce chemicals which inhibit the growth of competitors, including pathogenic organisms.

In certain embodiments, the microorganism is unculturable. This should be taken to mean that the microorganism is not known to be culturable or is difficult to culture using methods known to one skilled in the art.

Microorganisms of the present disclosure may be collected or obtained from any source or contained within and/or associated with material collected from any source.

In one embodiment, a microorganism or a combination of microorganisms, may provide likely or predicted benefit to a plant. For example, the microorganism may be predicted to: improve nitrogen fixation; release phosphate from the soil organic matter; release phosphate from the inorganic forms of phosphate (e.g., rock phosphate); “fix carbon” in the root microsphere; live in the rhizosphere of the plant thereby assisting the plant in absorbing nutrients from the surrounding soil and then providing these more readily to the plant; increase the number of nodules on the plant roots and thereby increase the number of symbiotic nitrogen fixing bacteria (e.g., Rhizobium species) per plant and the amount of nitrogen fixed by the plant; elicit plant defensive responses such as ISR (induced systemic resistance) or SAR (systemic acquired resistance) which help the plant resist the invasion and spread of pathogenic microorganisms; compete with microorganisms deleterious to plant growth or health by antagonism, or competitive utilization of resources such as nutrients or space; change the color of one or more part of the plant, or change the chemical profile of the plant, its smell, taste or one or more other quality.

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either applied directly to the plant growth medium, or concentrated by filtering or centrifugation, diluted to an appropriate concentration and applied to the plant growth medium with the bulk of the salt removed. By way of further example, microorganisms from mineralized or toxic sources may be similarly treated to recover the microbes for application to the plant growth material to minimize the potential for damage to the plant.

In some embodiments, a mixed population of microorganisms is used in the methods of the disclosure.

### Genome Modification of Microbes

In some embodiments, the microorganism may have its genome altered in some way to provide an improved trait of interest, for example improvement of nitrogen fixation for non-leguminous crops.

Various methods are known in the art for modification of polynucleotides in a cell (which includes, without limitation, any polynucleotide sequence comprised within the cell, including genomic, chromosomal, and plasmid DNA). Briefly, a single- or double-strand break is introduced into a target polynucleotide (the subject of the modification), the result of which may be an insertion of at least one nucleotide, the deletion of at least one nucleotide, the replacement of at least one nucleotide, or any combination of the preceding, according to the desire of the practitioner.

The single- or double-strand break (SSB or DSB) may be accomplished by any of a number of methods, including the utilization of chemicals or radiation, a result of the process of homologous recombination, by the introduction of a specific or non-specific nuclease, or by any combination of the preceding.

Enzymes that effect polynucleotide cleavage are known in the art, and may include (without limitation): restriction endonucleases, meganucleases, TALENs, Zinc Fingers, or Cas endonucleases.

In some aspects, the present disclosure relates to isolated, genetically-modified microbes that have improved nitrogen fixing activity, as compared to non-genetically modified variants of the same species or strain of microbe.

Glutamine (Gln) is the universal nitrogen signal in all free-living diazotrophs (see, for example, Wang et al., PLOS Genetics, 2018). Gram Negative bacteria, such as Klebsiella and Pseudomonas, have well-elucidated nitrogen pathways, and have easier, more predictable gene delivery and expression for genome modified strains. In the Gram-Negative organism Klebsiella, NifL is the negative regulator of the nif operon. When intracellular glutamine is high (nitrogen excess), NifL forms a repressor complex to inactivate the nif operon expression.

In the Gram-Negative organism Azospirillum, NifA activates transcription of the nif operon. Expression of nifA is regulated by glutamine through ntrB phosphorylation of ntrC. Nitrogenase is inactivated pos-transcriptionally.

In contrast, Gram-Positive bacteria, such as Paenibacillus described herein, are more difficult to transform and have less-studied nitrogen fixation pathways.

Thus, successful genome modification that results in greater nitrogen fixation capability for a Gram-Positive bacterium like Paenibacillus is not only surprising, but greatly needed in agriculture biotechnology. Because of the spore-forming capabilities of Paenibacillus, there is increased commercial potential for a product comprising a gene-edited Paenibacillus strain that improves nitrogen fixation for crop plants.

In Gram-Positive bacteria, the nif operon controls the nitrogen fixation pathways through GlnR. Binding of GlnR to Site I activates Nif expression, while binding of GlnR to Site II represses Nif expression. Thus, a gene target for improving Nitrogen fixation in Paenibacillus is the Nif activator/repressor GlnR and its binding sites.

In Paenibacillus, the nif operon (cluster) comprises a number of genes, including: nifB, nifH, nifD, nifK, nifE, nifN, nifX, hesA and nifV. Although highly conserved among the N₂-fixing Paenibacillus strains, there are some variations in DNA sequences of the nif clusters, which can be divided to two sub-groups: Subgroup I and Subgroup II. The 9 genes nifBHDKENXhesAnifV of the nif gene cluster within Sub-group I are contiguous, while there is an ORF of 261-561 bp, whose predicted product is unknown, between nifX and hesA within Sub-group II. Paenibacillus species P. polymyxa and P. tritici are examples of Subgroup I. Paenibacillus species P. albidus, P. anaericanus, P. azotifigens, P. borealis, P. donghaensis, P. ehimensis, P. graminis, P. jilunlii, P. odorifer, P. panacisoli, P. phoenicis, P. pocheonensis, P. rhizoplanae, P. silage, P. taohuashanense, P. thermophilus, P. typhae, and P. wynnii are examples of Subgroup II. Within the Paenibacillus genus, are two distinct Subgroups, Subgroup I and Subgroup II, each comprising a different operon composition. Subgroup I Paenibacillus, such as Paenibacillus polymyxa, comprise in this order: nifB, nifH, nifD, nifK nifE, nifN, nifZ, hesA, nifV. Subgroup II Paenibacillus, such as Paenibacillus graminis, comprise in this order: nifB, nifH, nifD, nifK, nifE, nifN, nifX, orf1, hesA, nifV.

Most biological nitrogen fixation is catalyzed by molybdenum-dependent nitrogenase, which is distributed within bacteria and archaea. This enzyme is composed of two component proteins, MoFe protein and Fe protein. The MoFe protein component is an α2β2 heterotetramer (encoded by nifD and nifK) that contains two metalloclusters; FeMo-co, a [Mo-7Fe-9S-C-homocitrate] cluster which serves as the active site of substrate binding and reduction and the P-cluster, a [8Fe-7S] cluster which shuttles electrons to FeMo-co. The Fe protein (encoded by nifH) is a homodimer bridged by an intersubunit [4Fe-4S] cluster that serves as the obligate electron donor to the MoFe protein. The assembly pathway for the biosynthesis of nitrogenase is complex. Apart from the structural subunits encoded by nifH, nifD and nifK, several genes are required for the biosynthesis of the metalloclusters, in addition to other gene products necessary to produce a fully functional enzyme. It is now well established from genetic and biochemical analysis that nifE, nifN, nifX, nifB, nifQ, nifV, nifY, and nifH contribute to the synthesis and insertion of FeMo-co into nitrogenase, that nifU, nifS, and nifZ play an important role in synthesis of metalloclusters and that nifM is required for proper folder of nitrogenase Fe protein. (Wang et al., PLOS Genet. 2013 October; 9 (10): e1003865).

The nitrogenase enzyme complex consists of the following two conserved proteins: the MoFe protein, composed of subunits encoded by the nifD and nifK genes; and the Fe protein, encoded by the nifH gene. The nitrogenase iron protein gene, nifH, is one of the oldest existing functional genes in the history of gene evolution. The nucleotide sequences for coding regions of nifHDK genes among all nitrogen-fixing organisms are highly conserved. However, the copy numbers and arrangement of nifH, nifD, and nifK are different among the different diazotrophic bacteria.

Although the majority of present-day biological N2reduction is catalyzed by the Mo-nitrogenase, two homologous alternative nitrogenases: V- and Fe-nitrogenase are important biological sources of fixed nitrogen in environments where Mo is limiting. V- and Fe-nitrogenase are encoded by the vnf and anf genes. The Mo-, V- and Fe-nitrogenases are not equally distributed in nature. Most diazotrophs, such as K. pneumoniae, possesses only the Mo-nitrogenase, while some organisms, like A. vinelandii, possess all three types of nitrogenases, and other organisms, like Rhodobacter capsulatus and Rhodospirillum rubrum, carry the Mo- and Fe-nitrogenases (Xie et al., 2014, PLOS Genetics 10 (3): e1004231).

Editing targets of various polynucleotides in the genome of Paenibacillus can be selected to increase nitrogen fixation in the absence of exogenously-applied Nitrogen, in the presence of minimal added Nitrogen (e.g., ammonium), as well as in the presence of added Nitrogen.

A non-limiting selection of editing targets include: nif1, nif2, nif4, anf4, anf5, nifH, GlnR Binding Site II, glnR, cueR, nrgA, sodA, hesA2, katA, glnK, nifB.

### NifH

The nitrogenase enzyme complex consists of the following two conserved proteins: the MoFe protein, composed of subunits encoded by the nifD and nifK genes; and the Fe protein, encoded by the nifH gene. The nitrogenase iron protein gene, nifH, is one of the oldest existing functional genes in the history of gene evolution. The nucleotide sequences for coding regions of nifHDK genes among all nitrogen-fixing organisms are highly conserved. However, the copy numbers and arrangement of nifH, nifD, and nifK are different among the different diazotrophic bacteria.

### GlnR

In Paenibacillus bacteria, the nif operon controls the nitrogen fixation pathways through GlnR; nif operon gene transcription is regulated by ammonium and oxygen. Knockouts (removal of entire coding region ATG to M156, leaving the stop codon in the resulting sequence, by homologous recombination) and C25 truncations (removal DNA encoding the last 25 amino acids of glnR) of GlnR were created to assess impact on Nitrogen fixation.

### GlnR Binding Sites I and II

Binding of GlnR to Site I activates Nif expression, while binding of GlnR to Site II represses Nif expression. Further, GlnR has a higher affinity for binding to Site II.

### CueR

In many bacterial species, CueR is generally recognized as the regulator of the Cue copper efflux system, activating gene expression in response to high levels of intracellular copper. In our Paenibacillus polymyxa, the CueR ORF shares a bidirectional promoter with the ammonium transporter NrgA. CueR is HTH-type transcriptional regulator like GlnR, and its proximity to NrgA in Paenibacillus polymyxa indicates it may play a role in transcription of the ammonium transporter as well. If this is the case, removal of CueR misregulates the expression of NrgA relative to nitrogen levels, causing reduced transport of ammonium into the cell. Decreased levels of ammonium in the cell induces the cell to utilize atmospheric nitrogen through expression of nitrogenase.

hesA2

hesA2 encodes an enzyme that may be involved in shuttling molybdenum for cofactor biosynthesis.

katA

katA encodes a catalase which detoxifies hydrogen peroxide to water and oxygen.

sodA

sodA encodes superoxide dismutase which detoxifies superoxide radicals to oxygen and hydrogen peroxide.

glnK

glnK encodes a PII protein which senses cellular nitrogen status.

nifB

nifB encodes a SAM-dependent enzyme which catalyzes formation of Fe—S cofactor for nitrogenase.

nif1

nif1 encodes the full molybdenum-dependent nitrogenase enzyme, including genes required for the proper biosynthesis, folding, and cofactor insertion of the MoFe and Fe proteins. Is the primary enzyme responsible for conversion of atmospheric N2 to NH3.

nif2

nif2 encodes the structural subunits of molybdenum-dependent nitrogenase enzyme, including many, but not all, genes required for the proper biosynthesis, folding, and cofactor insertion of the MoFe and Fe proteins. Is the primary enzyme responsible for conversion of atmospheric N2 to NH3.

nif4

nif4 encodes the dinitrogenase reducatse NifH, which is the Fe protein of the molybdenum-dependent nitrogenase enzyme, and the radical S-adenosyl methionine enzyme NifB, which is involved in the biosynthesis of the FeMo-co component of the molybdenum-dependent nitrogenase enzyme. These gene products may complement nitrogenase activity in strains which already encode the other required genes of nitrogenase.

anf4

anf4 encodes the structural subunits of the iron-dependent nitrogenase enzyme, including the Fe and Fe—Fe proteins. Is an alternate enzyme to molybdenum-dependent nitrogenase for conversion of atmospheric N2 to NH3.

anf5

anf5 encodes the dinitrogenase reductase anfH, the Fe protein of the iron-dependent nitrogenase enzyme, an alternate enzyme to molybdenum-dependent nitrogenase for conversion of atmospheric N2 to NH3. This may complement nitrogenase activity in strains which already encode the other required genes of nitrogenase.

It would be appreciated by one of ordinary skill in the art that similar editing targets in other microorganisms, including other Gram-negative bacteria as well as Gram-positive bacteria (e.g., Klebsiella) are possible with the teachings of the instant disclosure.

### Microbial Consortia

In aspects, the disclosure provides microbial consortia comprising a combination of at least any two microbes, wherein one is a genetically modified strain comprising one or more edit described herein, for example a Paenibacillus edited strain described in Table 1. In some embodiments, the Paenibacillus strain is a species selected from the group consisting of: polymyxa, tritici, albidus, anaericanus, azotifigens, borealis, donghaensis, ehimensis, graminis, jilunlii, odorifer, panacisoli, phoenicis, pocheonensis, rhizoplanae, silage, taohuashanense, thermophilus, typhae, and wynnii. In some embodiments, the Paenibacillus strain is of Subgroup I. In some embodiments, the Paenibacillus strain is of Subgroup II.

In certain embodiments, the consortia of the present disclosure comprise two microbes, or three microbes, or four microbes, or five microbes, or six microbes, or seven microbes, or eight microbes, or nine microbes, or ten or more microbes. Said microbes of the consortia are different microbial species, or different strains of a microbial species.

### Microbial-Produced Compositions

In some cases, the microbes of the present disclosure may produce one or more compounds and/or have one or more activities, e.g., one or more of the following: production of a metabolite, production of a phytohormone such as auxin, production of acetoin, production of an antimicrobial compound, production of a siderophore, production of a polyketide, production of a phenazine, production of a cellulase, production of a pectinase, production of a chitinase, production of a glucanase, production of a xylanase or protease or organic acid or lipopeptide or polynucleotide or polypeptide, nitrogen fixation, mineral phosphate solubilization, or any combination and/or plurality of the preceding.

For example, a microbe of the disclosure may produce a phytohormone selected from the group consisting of an auxin, a cytokinin, a gibberellin, ethylene, a brassinosteroid, and abscisic acid.

Thus, a “metabolite produced by” a microbe of the disclosure, is intended to capture any molecule (small molecule, vitamin, mineral, protein, nucleic acid, lipid, fat, carbohydrate, etc.) produced by the microbe. Often, the exact mechanism of action, whereby a microbe of the disclosure imparts a beneficial trait upon a given plant species is not known. It is hypothesized, that in some instances, the microbe is producing a metabolite that is beneficial to the plant. Thus, in some aspects, a cell-free or inactivated preparation of microbes is beneficial to a plant, as the microbe does not have to be alive to impart a beneficial trait upon the given plant species, so long as the preparation includes a metabolite that was produced by said microbe and which is beneficial to a plant.

In one embodiment, the microbes of the disclosure may produce auxin (e.g., indole-3-acetic acid (IAA)). Production of auxin can be assayed. Many of the microbes described herein may be capable of producing the plant hormone auxin indole-3-acetic acid (IAA) when grown in culture. Auxin plays a key role in altering the physiology of the plant, including the extent of root growth.

Therefore, in an embodiment, the microbes of the disclosure are present as a population disposed on the surface or within a tissue of a given plant species. The microbes may produce a composition, such as a metabolite, in an amount effective to cause a detectable increase in the amount of composition that is found on or within the plant, when compared to a reference plant not treated with the microbes or cell-free or inactive preparations of the disclosure. The composition produced by said microbial population may be beneficial to the plant species.

Such microbial-produced compositions may be present in the cell culture broth or medium/a in which the microbes are grown, or may encompass an exudate produced by the microbes. As used herein, “exudate” refers to one or more compositions excreted by or extracted from one or more microbial cell(s). As used herein, “broth” refers to the collective composition of a cell culture medium after microbial cells are placed in the medium. The composition of the broth may change over time, during different phases of microbial growth and/or development. Broth and/or exudate may improve the traits of plants with which they become associated.

### Microbial-Induced Traits in Plants

The present disclosure utilizes microbes to impart beneficial properties (or beneficial traits) to desirable plant species, such as agronomic species of interest. In the current disclosure, the terminology “beneficial property”, “beneficial trait”, or “trait of interest”, is used interchangeably and denotes that a desirable plant phenotypic or genetic property of interest is modulated, by the application of a microbe or microbial consortia as described herein. As aforementioned, in some aspects, it may very well be that a metabolite produced by a given microbe is ultimately responsible for modulating or imparting a beneficial trait to a given plant.

There are a vast number of beneficial traits that can be modulated by the application of microbes of the disclosure. For instance, the microbes may have the ability to impart one or more beneficial properties to a plant species, for example: increased growth, increased yield, increased nitrogen utilization efficiency, increased stress tolerance, increased drought tolerance, increased photosynthetic rate, enhanced water use efficiency, increased pathogen resistance, modifications to plant architecture that don't necessarily impact plant yield, but rather address plant functionality, causing the plant to increase production of a metabolite of interest, etc.

In aspects, the microbes taught herein provide a wide range of agricultural applications, including: improvements in yield of grain, fruit, and flowers, improvements in growth of plant parts, improved ability to utilize nutrients (e.g., nitrogen, phosphate, and the like), improved resistance to disease, biopesticidal effects including improved resistance to fungi, insects, and/or nematodes; improved survivability in extreme climate, and improvements in other desired plant phenotypic characteristics.

In some aspects, the isolated microbes, consortia, and/or agricultural compositions of the disclosure can be applied to a plant, in order to modulate or alter a plant characteristic such as altered oil content, altered protein content, altered seed carbohydrate composition, altered seed oil composition, altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved nutrient utilization (e.g., phosphate, potassium, and the like), improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, reduced pathogen levels (e.g., via the excretion of metabolites that impair pathogen survival), pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome relative to a reference plant.

In some aspects, the isolated microbes, consortia, and/or agricultural compositions of the disclosure can be applied to a plant, in order to modulate in a negative way, a particular plant characteristic. For example, in some aspects, the microbes of the disclosure are able to decrease a phenotypic trait of interest, as this functionality can be desirable in some applications. For instance, the microbes of the disclosure may possess the ability to decrease root growth or decrease root length. Or the microbes may possess the ability to decrease shoot growth or decrease the speed at which a plant grows, as these modulations of a plant trait could be desirable in certain applications.

In some embodiments, the isolated microbes, consortia, and/or agricultural compositions of the disclosure can be applied to a plant, in order to impart nematode stress tolerance to plants.

In some embodiments, the isolated microbes, consortia, and/or agricultural compositions of the disclosure can be applied to a plant, in order to provide biostimulation (biostimulant effects) to plants. In some embodiments, the isolated microbes, consortia, and/or agricultural compositions of the disclosure can be applied to a plant, in order to provide disease tolerance to plants.

### Agricultural Compositions

In some embodiments, the microbes of the disclosure are combined with agricultural compositions. Agricultural compositions generally refer to organic and inorganic compounds that can include compositions that promote the cultivation of the microbe and/or the plant element; compositions involved in formulation of microbes for application to plant elements (for example, but not limited to: wetters, compatibilizing agents (also referred to as “compatibility agents”), antifoam agents, cleaning agents, sequestering agents, drift reduction agents, neutralizing agents and buffers, corrosion inhibitors, dyes, odorants, spreading agents (also referred to as “spreaders”), penetration aids (also referred to as “penetrants”), sticking agents (also referred to as “stickers” or “binders”), dispersing agents, thickening agents (also referred to as “thickeners”), stabilizers, emulsifiers, freezing point depressants, antimicrobial agents, and the like); compositions involved in conferring protection to the plant element or plant (for example, but not limited to: pesticides, nematicides, fungicides, bactericides, herbicides, and the like); as well as other compositions that may be of interest for the particular application.

In some embodiments, the agricultural compositions of the present disclosure are solid. Where solid compositions are used, it may be desired to include one or more carrier materials with the active isolated microbe or consortia. In some embodiments, the present disclosure teaches the use of carriers including, but not limited to: mineral earths such as silicas, silica gels, silicates, talc, kaolin, attaclay, limestone, chalk, loess, clay, dolomite, diatomaceous earth, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or compositions of these.

### Growth Compositions

In some embodiments, a composition is provided to the microbe and/or the plant element that promotes the growth and development. Exemplary compositions include liquid (such as broth, media) and/or solid (such as soil, nutrients). Various organic or inorganic compounds may be added to the growth composition to facilitate the health of the microbe, alone or in combination with the plant element, for example but not limited to: amino acids, vitamins, minerals, carbohydrates, simple sugars, lipids.

### Formulation Compositions

One or more compositions, in addition to the microbe(s) or microbial-produced composition, may be combined for various application, stability, activity, and/or storage reasons. The additional compositions may be referred to as “formulation components”.

In some embodiments, the agricultural compositions disclosed herein may be formulated as a liquid, a solid, a gas, or a gel.

Thus in some embodiments, the present disclosure teaches that the agricultural compositions disclosed herein can include compounds or salts such as monoethanolamine salt, sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate.

In some embodiments, the present disclosure teaches that agricultural compositions can include binders such as: polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or compositions of these; lubricants such as magnesium stearate, sodium stearate, talc or polyethylene glycol, or compositions of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or compositions of these.

In some embodiments, the agricultural compositions comprise surface-active agents. In some embodiments, the surface-active agents are added to liquid agricultural compositions. In other embodiments, the surface-active agents are added to solid formulations, especially those designed to be diluted with a carrier before application. Thus, in some embodiments, the agricultural compositions comprise surfactants. Surfactants are sometimes used, either alone or with other additives, such as mineral or vegetable oils as adjuvants to spray-tank mixes to improve the biological performance of the microbes on the target. The types of surfactants used for bioenhancement depend generally on the nature and mode of action of the microbes. The surface-active agents can be anionic, cationic, or nonionic in character, and can be employed as emulsifying agents, wetting agents, suspending agents, or for other purposes. In some embodiments, the surfactants are non-ionics such as: alky ethoxylates, linear aliphatic alcohol ethoxylates, and aliphatic amine ethoxylates. Surfactants conventionally used in the art of formulation and which may also be used in the present formulations are described, in McCutcheon's Detergents and Emulsifiers Annual, MC Publishing Corp., Ridgewood, N.J., 1998, and in Encyclopedia of Surfactants, Vol. I-III, Chemical Publishing Co., New York, 1980-81. In some embodiments, the present disclosure teaches the use of surfactants including alkali metal, alkaline earth metal or ammonium salts of aromatic sulfonic acids, for example, ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or compositions of these.

In some embodiments, the present disclosure teaches other suitable surface-active agents, including salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutyl-naphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, particularly methyl esters.

In some embodiments, the agricultural compositions comprise wetting agents. A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank or other vessel to reduce the wetting time of wettable powders and to improve the penetration of water into water-dispersible granules. In some embodiments, examples of wetting agents used in the agricultural compositions of the present disclosure, including wettable powders, suspension concentrates, and water-dispersible granule formulations are: sodium lauryl sulphate; sodium dioctyl sulphosuccinate; alkyl phenol ethoxylates; and aliphatic alcohol ethoxylates.

In some embodiments, the agricultural compositions of the present disclosure comprise dispersing agents. A dispersing agent is a substance which adsorbs onto the surface of particles and helps to preserve the state of dispersion of the particles and prevents them from re-aggregating. In some embodiments, dispersing agents are added to agricultural compositions of the present disclosure to facilitate dispersion and suspension during manufacture, and to ensure the particles redisperse into water in a spray tank. In some embodiments, dispersing agents are used in wettable powders, suspension concentrates, and water-dispersible granules. Surfactants that are used as dispersing agents have the ability to adsorb strongly onto a particle surface and provide a charged or steric barrier to re-aggregation of particles. In some embodiments, the most commonly used surfactants are anionic, non-ionic, or mixtures of the two types.

In some embodiments, for wettable powder formulations, the most common dispersing agents are sodium lignosulphonates. In some embodiments, suspension concentrates provide very good adsorption and stabilization using polyelectrolytes, such as sodium naphthalene sulphonate formaldehyde condensates. In some embodiments, tristyrylphenol ethoxylate phosphate esters are also used. In some embodiments, such as alkylarylethylene oxide condensates and EO-PO block copolymers are sometimes combined with anionics as dispersing agents for suspension concentrates.

In some embodiments, the agricultural compositions of the present disclosure comprise polymeric surfactants. In some embodiments, the polymeric surfactants have very long hydrophobic ‘backbones’ and a large number of ethylene oxide chains forming the ‘teeth’ of a ‘comb’ surfactant. In some embodiments, these high molecular weight polymers can give very good long-term stability to suspension concentrates, because the hydrophobic backbones have many anchoring points onto the particle surfaces. In some embodiments, examples of dispersing agents used in agricultural compositions of the present disclosure are: sodium lignosulphonates; sodium naphthalene sulphonate formaldehyde condensates; tristyrylphenol ethoxylate phosphate esters; aliphatic alcohol ethoxylates; alky ethoxylates; EO-PO block copolymers; and graft copolymers.

In some embodiments, the agricultural compositions of the present disclosure comprise emulsifying agents. An emulsifying agent is a substance, which stabilizes a suspension of droplets of one liquid phase in another liquid phase. Without the emulsifying agent the two liquids would separate into two immiscible liquid phases. In some embodiments, the most commonly used emulsifier blends include alkylphenol or aliphatic alcohol with 12 or more ethylene oxide units and the oil-soluble calcium salt of dodecylbenzene sulphonic acid. A range of hydrophile-lipophile balance (“HLB”) values from 8 to 18 will normally provide good stable emulsions. In some embodiments, emulsion stability can sometimes be improved by the addition of a small amount of an EO-PO block copolymer surfactant.

In some embodiments, the agricultural compositions of the present disclosure comprise solubilizing agents. A solubilizing agent is a surfactant, which will form micelles in water at concentrations above the critical micelle concentration. The micelles are then able to dissolve or solubilize water-insoluble materials inside the hydrophobic part of the micelle. The types of surfactants usually used for solubilization are non-ionics: sorbitan monooleates; sorbitan monooleate ethoxylates; and methyl oleate esters.

In some embodiments, the agricultural compositions of the present disclosure comprise organic solvents. Organic solvents are used mainly in the formulation of emulsifiable concentrates, ULV formulations, and to a lesser extent granular formulations. Sometimes mixtures of solvents are used. In some embodiments, the present disclosure teaches the use of solvents including aliphatic paraffinic oils such as kerosene or refined paraffins. In other embodiments, the present disclosure teaches the use of aromatic solvents such as xylene and higher molecular weight fractions of C9 and C10 aromatic solvents. In some embodiments, chlorinated hydrocarbons are useful as co-solvents to prevent crystallization of pesticides when the formulation is emulsified into water. Alcohols are sometimes used as co-solvents to increase solvent power.

In some embodiments, the agricultural compositions comprise gelling agents. Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. In some embodiments, the agricultural compositions comprise one or more thickeners including, but not limited to: montmorillonite, e.g., bentonite; magnesium aluminum silicate; and attapulgite. In some embodiments, the present disclosure teaches the use of polysaccharides as thickening agents. The types of polysaccharides most commonly used are natural extracts of seeds and seaweeds or synthetic derivatives of cellulose. Some embodiments utilize xanthan and some embodiments utilize cellulose. In some embodiments, the present disclosure teaches the use of thickening agents including, but are not limited to: guar gum; locust bean gum; carrageenan; alginates; methyl cellulose; sodium carboxymethyl cellulose (SCMC); hydroxyethyl cellulose (HEC). In some embodiments, the present disclosure teaches the use of other types of anti-settling agents such as modified starches, polyacrylates, polyvinyl alcohol, and polyethylene oxide. Another good anti-settling agent is xanthan gum.

In some embodiments, the presence of surfactants, which lower interfacial tension, can cause water-based formulations to foam during mixing operations in production and in application through a spray tank. Thus, in some embodiments, in order to reduce the tendency to foam, anti-foam agents are often added either during the production stage or before filling into bottles/spray tanks. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

In some embodiments, the agricultural compositions comprise a preservative.

In some embodiments, the agricultural compositions may be formulated as: a soil drench, a foliar spray, a dip treatment, an in-furrow treatment, a soil amendment, granules, a broadcast treatment, a post-harvest disease control treatment, or a seed treatment. In some embodiments, the agricultural compositions may be applied alone in or in rotation spray programs with other agricultural products.

In some embodiments, the agricultural compositions may be compatible with tank mixing. In some embodiments, the agricultural compositions may be compatible with tank mixing with other agricultural products. In some embodiments, the agricultural compositions may be compatible with equipment used for ground, aerial, and irrigation applications.

In some embodiments, the agricultural compositions may be applied to genetically modified seeds or plants.

### Protective Compositions

Further, the individual microbes, or microbial consortia, or microbial communities, developed according to the disclosed methods can be combined with known actives available in the agricultural space, such as: pesticide, herbicide, bactericide, fungicide, insecticide, virucide, miticide, nematicide, acaricide, plant growth regulator, rodenticide, anti-algae agent, biocontrol or beneficial agent. Further, the microbes, microbial consortia, or microbial communities developed according to the disclosed methods can be combined with known fertilizers. Such combinations may exhibit synergistic properties. Further still, the individual microbes, or microbial consortia, or microbial communities, developed according to the disclosed methods can be combined with inert ingredients. Also, in some aspects, the disclosed microbes are combined with biological active agents.

In some embodiments, the individual microbes, or microbial consortia, or microbial communities, developed according to the disclosed methods can be combined with biopesticides that function as an herbicide, bactericide, fungicide, insecticide, virucide, miticide, nematicide, acaricide, rodenticide, and/or anti-algae agent. Such biopesticides may be, but are not limited to, macrobial organisms (e.g., beneficial nematodes and the like), microbial organisms (e.g., Serenade, Bt, and the like), plant extracts (e.g., Timorex Gold and the like), biochemical (e.g., insect pheromones and the like), and/or minerals and oils (e.g., canola oil).

### Pesticides and Biopesticides

In some embodiments, the agricultural compositions of the present disclosure comprise pesticides, used in combination with the taught microbes. In some embodiments, the agricultural compositions of the present disclosure comprise biopesticides, used in combination with the taught microbes.

In some embodiments, the individual microbes, or microbial consortia, or microbial communities, developed according to the disclosed methods can be combined with known pesticides in the agricultural space, such as: pesticides that function as an herbicide, bactericide, fungicide, insecticide, virucide, miticide, nematicide, acaricide, rodenticide, and/or anti-algae agent.

In some embodiments, the individual microbes, or microbial consortia, or microbial communities, developed according to the disclosed methods can be combined with known biopesticides in the agricultural space, such as: biopesticides that function as an herbicide, bactericide, fungicide, insecticide, virucide, miticide, nematicide, acaricide, rodenticide, and/or anti-algae agent.

For example, in some embodiments, the present disclosure teaches agricultural compositions comprising one or more of the following active ingredients including: macrobial organisms (e.g., beneficial nematodes and the like), microbial organisms (e.g., Serenade, Bt, and the like), plant extracts (e.g., Timorex Gold and the like), biochemical (e.g., insect pheromones and the like), and/or minerals and oils (e.g., canola oil).

In some embodiments, the individual microbes, or microbial consortia, or microbial communities, developed according to the disclosed methods can be combined with an herbicide selected from the group consisting of: an acetamide selected from the group consisting of acetochlor, alachlor, butachlor, dimethachlor, dimethenamid, flufenacet, mefenacet, metolachlor, metazachlor, napropamide, naproanilide, pethoxamid, pretilachlor, propachlor, and thenylchlor; an amino acid derivative selected from the group consisting of bilanafos, glufosinate, and sulfosate; an aryloxyphenoxypropionate selected from the group consisting of clodinafop, cyhalofop-butyl, fenoxaprop, fluazifop, haloxyfop, metamifop, propaquizafop, quizalofop, and quizalo-fop-P-tefuryl; diquat and paraquat; a (thio)carbamate selected from the group consisting of asulam, butylate, carbetamide, desmedipham, dimepiperate, eptam (EPTC), esprocarb, molinate, orbencarb, phenmedipham, prosulfocarb, pyributicarb, thiobencarb, and triallate; a cyclohexanedione selected from the group consisting of butroxydim, clethodim, cycloxydim, profoxydim, sethoxydim, tepraloxydim, and tralkoxydim; a dinitroaniline selected from the group consisting of benfluralin, ethalfluralin, oryzalin, pendimethalin, prodiamine, and trifluralin; a diphenyl ether selected from the group consisting of acifluorfen, aclonifen, bifenox, diclofop, ethoxyfen, fomesafen, lactofen, and oxyfluorfen; a hydroxybenzonitrile selected from the group consisting of bomoxynil, dichlobenil, and ioxynil; an imidazolinone selected from the group consisting of imazamethabenz, imazamox, imazapic, imazapyr, imazaquin, and imazethapyr; a phenoxy acetic acid selected from the group consisting of clomeprop, 2,4-dichlorophenoxyacetic acid (2,4-D), 2,4-DB, dichlorprop, MCPA, MCPA-thioethyl, MCPB, and Mecoprop; a pyrazine selected from the group consisting of chloridazon, flufenpyr-ethyl, fluthiacet, norflurazon, and pyridate; a pyridine selected from the group consisting of aminopyralid, clopyralid, diflufenican, dithiopyr, fluridone, fluroxypyr, picloram, picolinafen, and thiazopyr; a sulfonyl urea selected from the group consisting of amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, and 14(2-chloro-6-propyl-imidazol[1,2]-blpyridazin-3-yl)sulfonyl)-3-(4,6-dimethoxy-pyrimidin-2-yl)urea; a triazine selected from the group consisting of ametryn, atrazine, cyanazine, a dimethametryn, ethiozin, hexazinone, metamitron, metribuzin, prometryn, simazine, terbuthylazine, terbutryn, and triaziflam; a urea compound selected from the group consisting of chlorotoluron, daimuron, diuron, fluometuron, isoproturon, linuron, methabenzthiazuron, and tebuthiuron; an acetolactate synthase inhibitor selected from the group consisting of bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, and pyroxsulam; and a compound selected from the group consisting of amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclol[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxyl]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

In some embodiments, the individual microbes, or microbial consortia, or microbial communities, developed according to the disclosed methods can be combined with an insecticide selected from the group consisting of: an organo(thio)phosphate selected from the group consisting of acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon; a carbamate selected from the group consisting of alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate; a pyrethroid selected from the group consisting of allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, taufluvalinate, tefluthrin, tetramethrin, tralomethrin, transfluthrin, profluthrin, and dimefluthrin; an insect growth regulator selected from the group consisting of a) a chitin synthesis inhibitor wherein said chitin synthesis inhibitor is a benzoylurea selected from the group consisting of chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, and clofentazine; b) an ecdysone antagonist selected from the group consisting of halofenozide, methoxyfenozide, tebufenozide, and azadirachtin; c) a juvenoid selected from the group consisting of pyriproxyfen, methoprene, and fenoxycarb; or d) a lipid biosynthesis inhibitor selected from the group consisting of spirodiclofen, spiromesifen, and spirotetramat; a nicotinic receptor agonist/antagonist compound selected from the group consisting of clothianidin, dinotefuran, imidacloprid, thiamethoxam, nitenpyram, acetamiprid, thiacloprid, and 1-(2-chloro-thiazol-5-ylmethyl)-2-nitrimino-3,5-dimethyl-[1,3,5]triazinane; a GABA antagonist compound selected from the group consisting of endosulfan, ethiprole, fipronil, vaniliprole, pyrafluprole, pyriprole, and 5-amino-1-(2,6-dichloro-4-methyl-phenyl)-4-sulfinamoyl-1H-pyrazole-3-c arbothioic acid amide; a macrocyclic lactone insecticide selected from the group consisting of abamectin, emamectin, milbemectin, lepimectin, spinosad, and spinetoram; a mitochondrial electron transport inhibitor (METI) I acaricide selected from the group consisting of fenazaquin, pyridaben, tebufenpyrad, tolfenpyrad, and flufenerim; a METI II and III compound selected from the group consisting of acequinocyl, fluacyprim, and hydramethylnon; chlorfenapyr; an oxidative phosphorylation inhibitor selected from the group consisting of cyhexatin, diafenthiuron, fenbutatin oxide, and propargite; cryomazine; piperonyl butoxide; a sodium channel blocker selected from the group consisting of indoxacarb and metaflumizone; and a compound selected from the group consisting of benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon.

In some embodiments, the present invention teaches a synergistic use of the presently disclosed microbes or microbial consortia with known pesticides in the agricultural space, such as: pesticides that function as an herbicide, bactericide, fungicide, insecticide, virucide, miticide, nematicide, acaricide, rodenticide, and/or anti-algae agent.

In some embodiments, the present invention teaches a synergistic use of the presently disclosed microbes or microbial consortia with known biopesticides in the agricultural space, such as: biopesticides that function as an herbicide, bactericide, fungicide, insecticide, virucide, miticide, nematicide, acaricide, rodenticide, and/or anti-algae agent.

In some embodiments, when the microbe or microbial consortia identified according to the taught methods is combined with a pesticide one witnesses an additive effect on a plant phenotypic trait of interest. In other embodiments, when the microbe or microbial consortia identified according to the taught methods is combined with a pesticide one witness a synergistic effect on a plant phenotypic trait of interest.

In some embodiments, when the microbe or microbial consortia identified according to the taught methods is combined with a biopesticide one witnesses an additive effect on a plant phenotypic trait of interest. In other embodiments, when the microbe or microbial consortia identified according to the taught methods is combined with a biopesticide one witnesses a synergistic effect on a plant phenotypic trait of interest.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agriculturally active pesticide compounds and also agricultural auxiliary pesticide compounds.

The isolated microbes and consortia of the present disclosure can synergistically increase the effectiveness of agriculturally active biopesticide compounds and also agricultural auxiliary biopesticide compounds.

### Plant Growth Regulators and Biostimulants

In some embodiments, the agricultural compositions of the present disclosure comprise plant growth regulators and/or biostimulants, used in combination with the taught microbes.

In some embodiments, the individual microbes, or microbial consortia, or microbial communities, developed according to the disclosed methods can be combined with known plant growth regulators in the agricultural space, such as: auxins, gibberellins, cytokinins, ethylene generators, growth inhibitors, and growth retardants.

For example, in some embodiments, the present disclosure teaches agricultural compositions comprising one or more of the following active ingredients including: ancymidol, butralin, alcohols, chloromequat chloride, cytokinin, daminozide, ethepohon, flurprimidol, giberrelic acid, gibberellin mixtures, indole-3-butryic acid (IBA), maleic hydrazide, mefludide, mepiquat chloride, mepiquat pentaborate, naphthalene-acetic acid (NAA), 1-napthaleneacetemide, (NAD), n-decanol, placlobutrazol, prohexadione calcium, trinexapac-ethyl, uniconazole, salicylic acid, abscisic acid, ethylene, brassinosteroids, jasmonates, polyamines, nitric oxide, strigolactones, or karrikins among others.

In some embodiments, the individual microbes, or microbial consortia, or microbial communities, developed according to the disclosed methods can be combined with seed inoculants known in the agricultural space, such as: QUICKROOTS®, VAULT®, RHIZO-STICK®, NODULATOR®, DORMAL®, SABREX®, among others. In some embodiments, a Bradyrhizobium inoculant is utilized in combination with any single microbe or microbial consortia disclosed here. In particular aspects, a synergistic effect is observed when one combines one of the aforementioned inoculants, e.g., QUICKROOTS® or Bradyrhizobium, with a microbe or microbial consortia as taught herein.

In some embodiments, the agricultural compositions of the present disclosure comprise a plant growth regulator, which contains: kinetin, gibberellic acid, and indole butyric acid, along with copper, manganese, and zinc.

In some embodiments, the present disclosure teaches agricultural compositions comprising one or more commercially available plant growth regulators, including but not limited to: Abide®, A-Rest®, Butralin®, Fair®, Royaltac M®, Sucker-Plucker®, Off-Shoot®, Contact-85®, Citadel®, Cycocel®, E-Pro®, Conklin®, Culbac®, Cytoplex®, Early Harvest®, Foli-Zyme®, Goldengro®, Happygro®, Incite®, Megagro®, Ascend®, Radiate®, Stimulate®, Suppress®, Validate®, X-Cyte®, B-Nine®, Compress®, Dazide®, Boll Buster®, BollD®, Cerone®, Cotton Quik®, Ethrel®, Finish®, Flash®, Florel®, Mature®, MFX®, Prep®, Proxy®, Quali-Pro®, SA-50®, Setup®, Super Boll®, Whiteout®, Cutless®, Legacy®, Mastiff®, Topflor®, Ascend®, Cytoplex®, Ascend®, Early Harvest®, Falgro®, Florgib®, Foli-Zyme®, GA3®, GibGro®, Green Sol®, Incite®, N-Large®, PGR IVR, Pro-Gibb®, Release®, Rouse®, Ryzup®, Stimulate®, BVB®, Chrysal®, Fascination®, Procone®, Fair®, Rite-Hite®, Royal®, Sucker Stuff®, Embark®, Sta-Lo®, Pix®, Pentia®, DipN Grow®, Goldengro®, Hi-Yield®, Rootone®, Antac®, FST-7®, Royaltac®, Bonzi®, Cambistat®, Cutdown®, Downsize®, Florazol®, Paclo®, Paczol®, Piccolo®, Profile®, Shortstop®, Trimmit®, Turf Enhancer® Apogee®, Armor Tech®, Goldwing®, Governor®, Groom®, Legacy®, Primeraone®, Primo®, Provair®, Solace®, T-Nex®, T-Pac®, Concise®, and Sumagic®.

In some embodiments, the present invention teaches a synergistic use of the presently disclosed microbes or microbial consortia with plant growth regulators and/or stimulants such as phytohormones or chemicals that influence the production or disruption of plant growth regulators.

In some embodiments, the present invention teaches that phytohormones can include: Auxins (e.g., Indole acetic acid IAA), Gibberellins, Cytokinins (e.g., Kinetin), Abscisic acid, Ethylene (and its production as regulated by ACC synthase and disrupted by ACC deaminase).

In some embodiments, the individual microbes, or microbial consortia, or microbial communities, developed according to the disclosed methods can be combined with biostimulants. Such biostimulants may be, but are not limited to, microbial organisms, plant extracts, seaweeds, acids, biochar, and the like.

In some embodiments, the individual microbes, or microbial consortia, or microbial communities, developed according to the disclosed methods can be combined with fertilizers, which may be organic (e.g., manure, blood, fish, and the like), nitrogen-based (e.g., nitrate, ammonium, urea, and the like), phosphate, and potassium. Such fertilizers may also contain micronutrients including, but not limited to, sulfur, iron, zinc, and the like.

In some embodiments, the present invention teaches additional plant-growth promoting chemicals that may act in synergy with the microbes and microbial consortia disclosed herein, such as: humic acids, fulvic acids, amino acids, polyphenols and protein hydrolysates.

Thus, in some embodiments, the disclosure provides for the application of the taught microbes in combination with Ascend® upon any crop. Further, the disclosure provides for the application of the taught microbes in combination with Ascend® upon any crop and utilizing any method or application rate.

In some embodiments, the present disclosure teaches agricultural compositions with biostimulants.

As used herein, the term “biostimulant” refers to any substance that acts to stimulate the growth of microorganisms that may be present in soil or other plant growing medium.

The level of microorganisms in the soil or growing medium is directly correlated to plant health. Microorganisms feed on biodegradable carbon sources, and therefore plant health is also correlated with the quantity of organic matter in the soil. While fertilizers provide nutrients to feed and grow plants, in some embodiments, biostimulants provide biodegradable carbon, e.g., molasses, carbohydrates, e.g., sugars, to feed and grow microorganisms. Unless clearly stated otherwise, a biostimulant may comprise a single ingredient, or a combination of several different ingredients, capable of enhancing microbial activity or plant growth and development, due to the effect of one or more of the ingredients, either acting independently or in combination.

In some embodiments, biostimulants are compounds that produce non-nutritional plant growth responses. In some embodiments, many important benefits of biostimulants are based on their ability to influence hormonal activity. Hormones in plants (phytohormones) are chemical messengers regulating normal plant development as well as responses to the environment. Root and shoot growth, as well as other growth responses are regulated by phytohormones. In some embodiments, compounds in biostimulants can alter the hormonal status of a plant and exert large influences over its growth and health. Thus, in some embodiments, the present disclosure teaches sea kelp, humic acids, fulvic acids, and B Vitamins as common components of biostimulants. In some embodiments, the biostimulants of the present disclosure enhance antioxidant activity, which increases the plant's defensive system. In some embodiments, vitamin C, vitamin E, and amino acids such as glycine are antioxidants contained in biostimulants.

In other embodiments, biostimulants may act to stimulate the growth of microorganisms that are present in soil or other plant growing medium. Prior studies have shown that when certain biostimulants comprising specific organic seed extracts (e.g., soybean) were used in combination with a microbial inoculant, the biostimulants were capable of stimulating growth of microbes included in the microbial inoculant. Thus, in some embodiments, the present disclosure teaches one or more biostimulants that, when used with a microbial inoculant, is capable of enhancing the population of both native microbes and inoculant microbes. For a review of some popular uses of biostimulants, please see Calvo et al., 2014, Plant Soil 383:3-41.

### Combinations of Plant Elements, Microbes, and Agricultural Compositions

In some embodiments, the present disclosure teaches that the individual microbes, or microbial consortia, or microbial communities, or any combination of the preceding, for example comprising any one or a plurality of microorganisms that include a genome-edited Paenibacillus strain described herein, may be applied to a plant element, optionally in combination with any agricultural composition, for the improvement of a plant phenotype.

Isolated microbes or communities or consortia (generally “microbes” or “microbe”, interchangeably) may be applied to a heterologous plant element, creating a synthetic combination. Microbes are considered heterologous to a plant element if they are not normally associated with the plant element in nature, or if found, are applied in amounts different than that found in nature. In some embodiments, the microbes may be found naturally in one part of a plant but not another, and introduction of the microbes to another part of the plant is considered a heterologous association.

It is further contemplated that the microbe, either isolated or in combination with a plant or plant element, may be further associated with one or more agricultural compositions, such as those described above.

Synthetic combinations of microbes and plant elements, microbes and agricultural compositions, and microbes and plant elements and agricultural compositions are contemplated (generally “synthetic compositions”, compositions that comprise components not typically found associated in nature).

### Plant Element Treatments

In some embodiments, the present disclosure also concerns the discovery that treating plant elements before they are sown or planted with a combination of one or more of the microbes or agricultural compositions of the present disclosure can enhance a desired plant trait, e.g., plant growth, plant health, and/or plant resistance to pests.

Thus, in some embodiments, the present disclosure teaches the use of one or more of the microbes or microbial consortia as plant element treatments. The plant element treatment can be a plant element coating applied directly to an untreated and “naked” plant element. However, the plant element treatment can be a plant element overcoat that is applied to a plant element that has already been coated with one or more previous plant element coatings or plant element treatments. The previous plant element treatments may include one or more active compounds, either chemical or biological, and one or more inert ingredients.

The term “plant element treatment” generally refers to application of a material to a plant element prior to or during the time it is planted in soil. Plant element treatment with microbes, and other agricultural compositions of the present disclosure, has the advantages of delivering the treatments to the locus at which the plant elements are planted shortly before germination of the plant element and emergence of a plant element.

In other embodiments, the present disclosure also teaches that the use of plant element treatments minimizes the amount of microbe or agricultural composition that is required to successfully treat the plants, and further limits the amount of contact of workers with the microbes and compositions compared to application techniques such as spraying over soil or over emerging plant element.

Moreover, in some embodiments, the present disclosure teaches that the microbes disclosed herein are important for enhancing the early stages of plant life (e.g., within the first thirty days following emergence of the plant element). Thus, in some embodiments, delivery of the microbes and/or compositions of the present disclosure as a plant element treatment places the microbe at the locus of action at a critical time for its activity.

In some embodiments, the microbial compositions of the present disclosure are formulated as a plant element treatment. In some embodiments, it is contemplated that the plant elements can be substantially uniformly coated with one or more layers of the microbes and/or agricultural compositions disclosed herein, using conventional methods of mixing, spraying, or a combination thereof through the use of treatment application equipment that is specifically designed and manufactured to accurately, safely, and efficiently apply plant element treatment products to plant elements. Such equipment uses various types of coating technology such as rotary coaters, drum coaters, fluidized bed techniques, spouted beds, rotary mists, or a combination thereof. Liquid plant element treatments such as those of the present disclosure can be applied via either a spinning “atomizer” disk or a spray nozzle, which evenly distributes the plant element treatment onto the plant element as it moves though the spray pattern. In aspects, the plant element is then mixed or tumbled for an additional period of time to achieve additional treatment distribution and drying.

The plant elements can be primed or unprimed before coating with the microbial compositions to increase the uniformity of germination and emergence. In an alternative embodiment, a dry powder formulation can be metered onto the moving plant element and allowed to mix until completely distributed.

In some embodiments, the plant elements have at least part of the surface area coated with a microbiological composition, according to the present disclosure. In some embodiments, a plant element coat comprising the microbial composition is applied directly to a naked plant element. In some embodiments, a plant element overcoat comprising the microbial composition is applied to a plant element that already has a plant element coat applied thereon. In some aspects, the plant element may have a plant element coat comprising, e.g., clothianidin and/or Bacillus firmus-I-1582, upon which the present composition will be applied on top of, as a plant element overcoat. In some aspects, the taught microbial compositions are applied as a plant element overcoat to plant elements that have already been treated with PONCHO™ VOTiVO™. In some aspects, the plant element may have a plant element coat comprising, e.g., Metalaxyl, and/or clothianidin, and/or Bacillus firmus-I-1582, upon which the present composition will be applied on top of, as a plant element overcoat. In some aspects, the taught microbial compositions are applied as a plant element overcoat to plant elements that have already been treated with ACCELERON™.

In some embodiments, the microorganism-treated plant elements have a microbial spore concentration, or microbial cell concentration, from about: 10\hat{ }2 to 10\hat{ }12, 10\hat{ }2 to 10\hat{ }11, 10\hat{ }2 to 10\hat{ }10, 10\hat{ }2 to 10\hat{ }9, 1\hat{ }02 to 10\hat{ }8, 10\hat{ }2 to 10\hat{ }7, 10\hat{ }2 to 10\hat{ }6, 10\hat{ }2 to 10\hat{ }5, 10\hat{ }2 to 10\hat{ }4, or 10\hat{ }2 to 10\hat{ }3 per plant element.

In some embodiments, the microorganism-treated plant elements have a microbial spore concentration, or microbial cell concentration, from about: 10\hat{ }3 to 10\hat{ }12, 10\hat{ }3 to 10\hat{ }11, 10\hat{ }3 to 10\hat{ }10, 10\hat{ }3 to 10\hat{ }9, 10\hat{ }3 to 10\hat{ }8, 10\hat{ }3 to 10\hat{ }7, 10\hat{ }3 to 10\hat{ }6, 10\hat{ }3 to 10\hat{ }5, or 10\hat{ }3 to 10\hat{ }4 per plant element.

In some embodiments, the microorganism-treated plant elements have a microbial spore concentration, or microbial cell concentration, from about: 10\hat{ }4 to 10\hat{ }12, 10\hat{ }4 to 10\hat{ }11, 10\hat{ }4 to 10\hat{ }10, 10\hat{ }4 to 10\hat{ }9, 10\hat{ }4 to 10\hat{ }8, 10\hat{ }4 to 10\hat{ }7, 10\hat{ }4 to 10\hat{ }6, or 10\hat{ }4 to 10\hat{ }5 per plant element.

In some embodiments, the microorganism-treated plant elements have a microbial spore concentration, or microbial cell concentration, from about: 10\hat{ }5 to 10\hat{ }12, 10\hat{ }5 to 10\hat{ }11, 10\hat{ }5 to 10\hat{ }10, 10\hat{ }5 to 10\hat{ }9, 10\hat{ }5 to 10\hat{ }8, 10\hat{ }5 to 10\hat{ }7, or 10\hat{ }5 to 10\hat{ }6 per plant element.

In some embodiments, the microorganism-treated plant elements have a microbial spore concentration, or microbial cell concentration, from about: 10\hat{ }5 to 10\hat{ }9 per plant element.

In some embodiments, the microorganism-treated plant elements have a microbial spore concentration, or microbial cell concentration, of at least about: 1×10\hat{ }3, or 1×10\hat{ }4, or 1×10\hat{ }5, or 1×10\hat{ }6, or 1×10\hat{ }7, or 1×10\hat{ }8, or 1×10\hat{ }9 per plant element.

In some embodiments, the amount of one or more of the microbes and/or agricultural compositions applied to the plant element depend on the final formulation, as well as size or type of the plant or plant element utilized. In some embodiments, one or more of the microbes are present in about 2% w/w/to about 80% w/w of the entire formulation. In some embodiments, the one or more of the microbes employed in the compositions is about 5% w/w to about 65% w/w, or 10% w/w to about 60% w/w by weight of the entire formulation.

In some embodiments, the plant elements may also have more spores or microbial cells per plant element, such as, for example about 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, 10\hat{ }15, 10\hat{ }16, or 10\hat{ }17 spores or cells per plant element.

In some embodiments, the plant element coats of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some embodiments, the plant element coats of the present disclosure can be 0.5 mm, 1 mm, 1.5 mm, 2 mm, 2.5 mm, 3 mm, 3.5 mm, 4 mm, 4.5 mm, or 5 mm thick.

In some embodiments, the plant element coats of the present disclosure can be at least 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, or 50% of the uncoated plant element weight.

In some embodiments, the microbial spores and/or cells can be coated freely onto the plant elements or they can be formulated in a liquid or solid composition before being coated onto the plant elements. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, it is contemplated that the solid or liquid microbial compositions of the present disclosure further contain functional agents e.g., activated carbon, nutrients (fertilizers), and other agents capable of improving the germination and quality of the products or a combination thereof.

Plant element coating methods and compositions that are known in the art can be particularly useful when they are modified by the addition of one of the embodiments of the present disclosure. Such coating methods and apparatus for their application are disclosed in, for example: U.S. Pat. Nos. 5,916,029; 5,918,413; 5,554,445; 5,389,399; 4,759,945; 4,465,017, and U.S. patent application Ser. No. 13/260,310, each of which is incorporated by reference herein.

Plant element coating compositions are disclosed in, for example: U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432, each of which is incorporated by reference herein.

In some embodiments, a variety of additives can be added to the plant element treatment formulations comprising the inventive compositions. Binders can be added and include those composed of an adhesive polymer that can be natural or synthetic without phytotoxic effect on the plant element to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorants may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, molybdenum and zinc.

A polymer or other dust control agent can be applied to retain the treatment on the plant element surface.

In some specific embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches. More examples can be found in, for example, U.S. Pat. No. 7,213,367, incorporated herein by reference.

Various additives, such as adherents, dispersants, surfactants, and nutrient and buffer ingredients, can also be included in the plant element treatment formulation. Other conventional plant element treatment additives include, but are not limited to: coating agents, wetting agents, buffering agents, and polysaccharides. At least one agriculturally acceptable carrier can be added to the plant element treatment formulation such as water, solids, or dry powders. The dry powders can be derived from a variety of materials such as calcium carbonate, gypsum, vermiculite, talc, humus, activated charcoal, and various phosphorous compounds.

In some embodiments, the plant element coating composition can comprise at least one filler, which is an organic or inorganic, natural or synthetic component with which the active components are combined to facilitate its application onto the plant element. In aspects, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

In some embodiments, the plant element treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the plant element, or alternatively may be added as part of the plant element coating composition of the disclosure.

In some embodiments, the formulation that is used to treat the plant element in the present disclosure can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation can be about 0.5% to about 99% by weight (w/w), or 5-40%, or as otherwise formulated by those skilled in the art.

As mentioned above, other conventional inactive or inert ingredients can be incorporated into the formulation. Such inert ingredients include, but are not limited to: conventional sticking agents; dispersing agents such as methylcellulose, for example, serve as combined dispersant/sticking agents for use in plant element treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the present disclosure can be found in Mccutcheon's, vol. 1, “Emulsifiers and Detergents,” MC Publishing Company, Glen Rock, N.J., U.S.A., 1996, incorporated by reference herein.

The plant element coating formulations of the present disclosure can be applied to plant elements by a variety of methods, including, but not limited to: mixing in a container (e.g., a bottle or bag), mechanical application, tumbling, spraying, and immersion. A variety of active or inert material can be used for contacting plant elements with microbial compositions according to the present disclosure.

In some embodiments, the amount of the microbes or agricultural composition that is used for the treatment of the plant element will vary depending upon the type of plant element and the type of active ingredients, but the treatment will comprise contacting the plant elements with an agriculturally effective amount of the inventive composition.

As discussed above, an effective amount means that amount of the inventive composition that is sufficient to affect beneficial or desired results. An effective amount can be administered in one or more administrations.

In some embodiments, in addition to the coating layer, the plant element may be treated with one or more of the following ingredients: other pesticides including fungicides and herbicides; herbicidal safeners; fertilizers and/or biocontrol agents. These ingredients may be added as a separate layer or alternatively may be added in the coating layer.

In some embodiments, the plant element coating formulations of the present disclosure may be applied to the plant elements using a variety of techniques and machines, such as fluidized bed techniques, the roller mill method, rotostatic plant element treaters, and drum coaters. Other methods, such as spouted beds may also be useful. The plant elements may be pre-sized before coating. After coating, the plant elements are typically dried and then transferred to a sizing machine for sizing. Such procedures are known in the art.

In some embodiments, the microorganism-treated plant elements may also be enveloped with a film overcoating to protect the coating. Such overcoatings are known in the art and may be applied using fluidized bed and drum film coating techniques.

In other embodiments of the present disclosure, compositions according to the present disclosure can be introduced onto a plant element by use of solid matrix priming. For example, a quantity of an inventive composition can be mixed with a solid matrix material and then the plant element can be placed into contact with the solid matrix material for a period to allow the composition to be introduced to the plant element. The plant element can then optionally be separated from the solid matrix material and stored or used, or the mixture of solid matrix material plus plant element can be stored or planted directly. Solid matrix materials which are useful in the present disclosure include polyacrylamide, starch, clay, silica, alumina, soil, sand, polyurea, polyacrylate, or any other material capable of absorbing or adsorbing the inventive composition for a time and releasing that composition into or onto the plant element. It is useful to make sure that the inventive composition and the solid matrix material are compatible with each other. For example, the solid matrix material should be chosen so that it can release the composition at a reasonable rate, for example over a period of minutes, hours, or days.

In some embodiments, the present disclosure teaches that the individual microbes, or microbial consortia, or microbial communities, developed according to the disclosed methods can be combined with any plant biostimulant.

In some embodiments, the present disclosure teaches agricultural compositions comprising one or more commercially available biostimulants, including but not limited to: Vitazyme®, Diehard™ Biorush®, Diehard™ Biorush® Fe, Diehard™ Soluble Kelp, Diehard™ Humate SP, Phocon®, Foliar Plus™, Plant Plus™, Accomplish LM®, Titan®, Soil Builder™, Nutri Life, Soil Solution™, Seed Coat™, PercPlus™, Plant Power®, CropKarb®, Thrust™, Fast2Grow®, Baccarat®, and Potente® among others.

In some embodiments, when the microbe or microbial consortia identified according to the taught methods is combined with an active chemical agent one witnesses an additive effect on a plant phenotypic trait of interest. In other embodiments, when the microbe or microbial consortia identified according to the taught methods is combined with an active chemical agent one witness a synergistic effect on a plant phenotypic trait of interest.

In some embodiments, when the microbe or microbial consortia identified according to the taught methods is combined with a fertilizer one witnesses an additive effect on a plant phenotypic trait of interest. In other embodiments, when the microbe or microbial consortia identified according to the taught methods is combined with a fertilizer one witness a synergistic effect on a plant phenotypic trait of interest.

In some embodiments, when the microbe or microbial consortia identified according to the taught methods is combined with a plant growth regulator, one witnesses an additive effect on a plant phenotypic trait of interest. In some embodiments, when the microbe or microbial consortia identified according to the taught methods is combined with a plant growth regulator, one witnesses a synergistic effect. In some aspects, the microbes of the present disclosure are combined with Ascend® and a synergistic effect is observed for one or more phenotypic traits of interest.

In some embodiments, when the microbe or microbial consortia identified according to the taught methods is combined with a biostimulant, one witnesses an additive effect on a plant phenotypic trait of interest. In some embodiments, when the microbe or microbial consortia identified according to the taught methods is combined with a biostimulant, one witnesses a synergistic effect.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agricultural active compounds and also agricultural auxiliary compounds.

In other embodiments, when the microbe or microbial consortia identified according to the taught methods is combined with a fertilizer one witnesses a synergistic effect.

Furthermore, in certain embodiments, the disclosure utilizes synergistic interactions to define microbial consortia. That is, in certain aspects, the disclosure combines together certain isolated microbial species, which act synergistically, into consortia that impart a beneficial trait upon a plant, or which are correlated with increasing a beneficial plant trait.

The agricultural compositions developed according to the disclosure can be formulated with certain auxiliaries, in order to improve the activity of a known active agricultural compound. This has the advantage that the amounts of active ingredient in the formulation may be reduced while maintaining the efficacy of the active compound, thus allowing costs to be kept as low as possible and any official regulations to be followed. In individual cases, it may also possible to widen the spectrum of action of the active compound since plants, where the treatment with a particular active ingredient without addition was insufficiently successful, can indeed be treated successfully by the addition of certain auxiliaries along with the disclosed microbial isolates and consortia. Moreover, the performance of the active may be increased in individual cases by a suitable formulation when the environmental conditions are not favorable.

Such auxiliaries that can be used in an agricultural composition can be an adjuvant. Frequently, adjuvants take the form of surface-active or salt-like compounds. Depending on their mode of action, they can roughly be classified as modifiers, activators, fertilizers, pH buffers, and the like. Modifiers affect the wetting, sticking, and spreading properties of a formulation. Activators break up the waxy cuticle of the plant and improve the penetration of the active ingredient into the cuticle, both short-term (over minutes) and long-term (over hours). Fertilizers such as ammonium sulfate, ammonium nitrate or urea improve the absorption and solubility of the active ingredient and may reduce the antagonistic behavior of active ingredients. pH buffers are conventionally used for bringing the formulation to an optimal pH.

In some embodiments, the plant element is a plant reproductive element (e.g., seed, tuber, bulb, and/or shoot). In some embodiments, the plant element is other than a plant reproductive element (e.g., leaf, stem, and/or root). In some embodiments, a plurality of plant elements are associated with the microbe(s) described herein.

In some embodiments, the plant or plant element becomes associated with one or more microbes described herein via an indirect method, such as but not limited to treatment of the growth medium in which the plant or plant element is placed.

For further embodiments of agricultural compositions of the present disclosure, See “Chemistry and Technology of Agrochemical Formulations,” edited by D. A. Knowles, copyright 1998 by Kluwer Academic Publishers, hereby incorporated by reference.

### Plants and Agronomic Benefits

A wide variety of plants, including those cultivated in agriculture, are capable of receiving benefit from the application of microbes, such as those described herein, including single microbes, consortia, and/or compositions produced therefrom, or comprising any of the preceding. Any number of a variety of different plants, including mosses and lichens and algae, may be used in the methods of the disclosure. In embodiments, the plants have economic, social, or environmental value. For example, the plants may include those used as: food crops, fiber crops, oil crops, in the forestry industry, in the pulp and paper industry, as a feedstock for biofuel production, and as ornamental plants.

The genetically modified microorganisms disclosed herein have application in the improvement of nitrogen fixation in plants. In some embodiments, such plants include those which lack natural nitrogen-fixing symbionts (e.g., non-leguminous crops), such as but not limited to: wheat, maize (corn), rice, and vegetables. In some embodiments, such plants include those that would benefit from additional nitrogen fixation.

### Methods of Application

The microorganisms may be applied to a plant, seedling, cutting, propagule, or the like and/or the growth medium containing said plant, using any appropriate technique known in the art.

However, by way of example, a microbe, consortium, or composition comprising the same, and/or a composition produced therefrom, may be applied to a plant, seedling, cutting, propagule, or the like, by spraying, coating, dusting, or any other method known in the art.

In another embodiment, the isolated microbe, consortia, or composition comprising the same may be applied directly to a plant seed prior to sowing.

In another embodiment, the isolated microbe, consortia, or composition comprising the same may applied directly to a plant seed, as a seed coating.

In one embodiment of the present disclosure, the isolated microbe, consortia, or composition comprising the same is supplied in the form of granules, or plug, or soil drench that is applied to the plant growth media.

In other embodiments, the isolated microbe, consortia, or composition comprising the same are supplied in the form of a foliar application, such as a foliar spray or liquid composition. The foliar spray or liquid application may be applied to a growing plant or to a growth media, e.g., soil.

In some embodiments, the isolated microbe, consortia, or composition comprising the same are supplied in a form selected from: a soil drench, a foliar spray, a dip treatment, an in furrow treatment, a soil amendment, granules, a broadcast treatment, a post-harvest disease control treatment, or a seed treatment. In some embodiments, the agricultural compositions may be applied alone in or in rotation spray programs.

In some embodiments, the isolated microbe, consortia, or composition comprising the same may be compatible with tank mixing. In some embodiments, the agricultural compositions may be compatible with tank mixing with other agricultural products. In some embodiments, the agricultural compositions may be compatible with equipment used for ground, aerial, and irrigation applications.

In another embodiment, the isolated microbe, consortia, or composition comprising the same may be formulated into granules and applied alongside seeds during planting. Or the granules may be applied after planting. Or the granules may be applied before planting.

In some embodiments, the isolated microbe, consortia, or composition comprising the same are administered to a plant or growth media as a topical application and/or drench application to improve crop growth, yield, and quality. The topical application may be via utilization of a dry mix or powder or dusting composition or may be a liquid based formulation.

In embodiments, the isolated microbe, consortia, or composition comprising the same can be formulated as: (1) solutions; (2) wettable powders; (3) dusting powders; (4) soluble powders; (5) emulsions or suspension concentrates; (6) seed dressings or coatings, (7) tablets; (8) water-dispersible granules; (9) water soluble granules (slow or fast release); (10) microencapsulated granules or suspensions; (11) as irrigation components, and (12) a component of fertilizers, pesticides, and other compatible amendments, among others. In in certain aspects, the compositions may be diluted in an aqueous medium prior to conventional spray application. The compositions of the present disclosure can be applied to the soil, plant, seed, rhizosphere, rhizosheath, or other area to which it would be beneficial to apply the microbial compositions. Further still, ballistic methods can be utilized as a means for introducing endophytic microbes.

In aspects, the compositions are applied to the foliage of plants. The compositions may be applied to the foliage of plants in the form of an emulsion or suspension concentrate, liquid solution, or foliar spray. The application of the compositions may occur in a laboratory, growth chamber, greenhouse, or in the field.

In another embodiment, microorganisms may be inoculated into a plant by cutting the roots or stems and exposing the plant surface to the microorganisms by spraying, dipping, or otherwise applying a liquid microbial suspension, or gel, or powder.

In another embodiment, the microorganisms may be injected directly into foliar or root tissue, or otherwise inoculated directly into or onto a foliar or root cut, or else into an excised embryo, or radicle, or coleoptile. These inoculated plants may then be further exposed to a growth media containing further microorganisms; however, this is not necessary.

In other embodiments, particularly where the microorganisms are unculturable, the microorganisms may be transferred to a plant by any one or a combination of grafting, insertion of explants, aspiration, electroporation, wounding, root pruning, induction of stomatal opening, or any physical, chemical or biological treatment that provides the opportunity for microbes to enter plant cells or the intercellular space. Persons of skill in the art may readily appreciate a number of alternative techniques that may be used.

In one embodiment, the microorganisms infiltrate parts of the plant such as the roots, stems, leaves and/or reproductive plant parts (become endophytic), and/or grow upon the surface of roots, stems, leaves and/or reproductive plant parts (become epiphytic) and/or grow in the plant rhizosphere. In one embodiment, the microorganisms form a symbiotic relationship with the plant.

While the invention has been particularly shown and described with reference to a preferred embodiment and various alternate embodiments, it will be understood by persons skilled in the relevant art that various changes in form and details can be made therein without departing from the spirit and scope of the invention. Various alterations, modifications, and improvements of the present disclosure that readily occur to those skilled in the art, including certain alterations, modifications, substitutions, and improvements are also part of this disclosure. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

All cited patents, patent applications, patent publications, and non-patent literature referred to in this application are herein incorporated by reference in their entirety, for all purposes, to the same extent as if each were individually and specifically incorporated by reference.

### EXAMPLES

The methods and compositions presented herein-based upon utilizing the disclosed isolated microbes, communities, consortia, and/or compositions comprising and/or produced by microbes or consortia or communities-improve one or more characteristics of plants, for example nitrogen fixation in agricultural crops.

The abbreviation “uL” means “microliters”, “ug” means “micrograms”.

### Example 1: Microbe Culture, Sequencing, and Target Selection

Paenibacillus strains were grown in culture media to obtain sufficient cellular growth.

A subsample of each of the strains was then aseptically transferred to nitrogen-free growth media and incubated under microaerophilic conditions for 72 hours.

Isolates of interest were grown to mid-log phase in R2A media. DNA was extracted with the Qiagen Powersoil DNA extraction kit and sequencing libraries were constructed with the iGenomix RipTide kit as per manufacturer instructions. Sequencing was performed on an Illumina HiSeq with PE150. Raw Illumina reads were trimmed to Q15 with Trimmomatic v38 (Bolger A M, Lohse M, and Usadel B. (2014). Trimmomatic: A flexible trimmer for Illumina Sequence Data. Bioinformatics, btu170) and assembled with SPAdes (Prjibelski A, Antipov D, Meleshko D, Lapidus A, and Korobeynikov A. (2020) Using SPAdes de novo assembler. Curr. Protoc. Bioinform. 70, e102) using default parameters. Assembled contigs were analyzed with BinSantity 0.5.4. (Graham E D, Heidelberg J F, and Tully B J. (2017) BinSanity: unsupervised clustering of environmental microbial assemblies using coverage and affinity propagation. PeerJ 5: e3035) for purity with a contamination cutoff of <5%. The largest bin was extracted and annotated with Prokka 1.8 (Seemann T. (2014) Prokka: rapid prokaryotic genome annotation. Bioinformatics 30 (14): 2068-9). Sequences of the 16S rDNA were identified by Prokka 1.8 and sequences were extracted directly from the .ffn file. Taxonomy was assigned by GTDB-tk using default parameters with the April 2021 database (Pierre-Alain Chaumeil, Aaron J Mussig, Philip Hugenholtz, Donovan H Parks, GTDB-Tk: a toolkit to classify genomes with the Genome Taxonomy Database, Bioinformatics, Volume 36, Issue 6, 15 Mar. 2020, Pages 1925-1927).

### Example 2: Editing of Paenibacillus Strains

Edits of Paenibacillus microbes to produce engineered strains that impart improved characteristics to plants with which they are associated may be accomplished by nucleotide insertion, nucleotide deletion, nucleotide replacement, and/or any combination or plurality of the preceding. The net effect may be one of upregulation, downregulation, knockout (of the target polynucleotide and/or the function of its encoded RNA or protein), and/or any combination or plurality of the preceding.

Because of the known challenges of working with Gram-positive bacteria such as Paenibacillus, as compared to more amenable Gram-negative bacteria such as Klebsiella, successful edits and successful associations with plants that impart improved benefits to the plants are surprising and unexpected.

Paenibacillus polymyxa Strain 77155 (NRRL Deposit No. B-68191 deposited 18 Aug. 2022) and Paenibacillus odorifer strain 17899 were used as exemplary editing strain for the methods described herein. It is appreciated that other microbes of the genus Paenibacillus as well as other genera and species, both Gram-positive and Gram-negative, may be used.

Editing targets and descriptions of modifications were accomplished as follows. Polynucleotide and polypeptide sequences may be obtained by any method known in the art. Edited strains with their edit types are given in Table 1. In some cases, the source of the component that was inserted (for example, on a replicative plasmid) was sourced from Strain 77155, or from strain 103408 (NRRL Deposit No. B-68113 deposited 7 Apr. 2022).

TABLE 1
Edits performed in Strains 77155 and 17899

| Strain | Modification(s) Present | | | | | | | | | | | | | | | | | |
|---|---|---|---|---|---|---|---|---|---|---|---|---|---|---|---|---|---|---|
| Designation | A | B | C | D | E | F | G | H | I | J | K | L | M | N | O | P | Q | R |
| 77155-G123 | X | | | | | | | | | | | | | | | | | |
| 77155-G169 | X | | | | | X | | | | | | | | | | | | |
| 77155-G168 | X | | | | | | X | | | | | | | | | | | |
| 77155-G170 | X | | | | | | | X | | | | | | | | | | |
| 77155-G171 | X | | | | | | | | X | | | | | | | | | |
| 77155-G172 | X | | | | | | | | | X | | | | | | | | |
| 77155-G122 | | X | | | | | | | | | | | | | | | | |
| 77155-G150 | | X | | | | X | | | | | | | | | | | | |
| 77155-G151 | | X | | | | | X | | | | | | | | | | | |
| 77155-G152 | | X | | | | | | X | | | | | | | | | | |
| 77155-G153 | | X | | | | | | | X | | | | | | | | | |
| 77155-G154 | | X | | | | | | | | X | | | | | | | | |
| 77155-G121 | | | X | | | | | | | | | | | | | | | |
| 77155-G124 | | | X | | | X | | | | | | | | | | | | |
| 77155-G127 | | | X | | | | X | | | | | | | | | | | |
| 77155-G128 | | | X | | | | | X | | | | | | | | | | |
| 77155-G129 | | | X | | | | | | X | | | | | | | | | |
| 77155-G130 | | | X | | | | | | | X | | | | | | | | |
| 77155-G182 | | | | X | | | | | | | | | | | | | | |
| 77155-G184 | | | | X | | X | | | | | | | | | | | | |
| 77155-G183 | | | | X | | | X | | | | | | | | | | | |
| 77155-G185 | | | | X | | | | X | | | | | | | | | | |
| 77155-G186 | | | | X | | | | | X | | | | | | | | | |
| 77155-G187 | | | | X | | | | | | X | | | | | | | | |
| 77155-G155 | | | | | X | | | | | | | | | | | | | |
| 77155-G156 | | | | | X | | X | | | | | | | | | | | |
| 77155-G157 | | | | | X | X | | | | | | | | | | | | |
| 77155-G158 | | | | | X | | | X | | | | | | | | | | |
| 77155-G159 | | | | | X | | | | X | | | | | | | | | |
| 77155-G160 | | | | | X | | | | | X | | | | | | | | |
| 77155-G189 | | | | | | | | | | | X | | | | | | | |
| 77155-G191 | | | | | | | | | | | | | | X | | | | |
| 77155-G192 | | | | | | | | | | | | | | | | X | | |
| 77155-G193 | | | | | | | | | | | | X | | | | | | |
| 77155-G194 | | | | | | | | | | | | | | | | | | X |
| 77155-G195 | | | | | | | | | | | | | | | X | | | |
| 77155-G196 | | | | | | | | | | | | | | | | | X | |
| 17899-G135 | | | | | | | | | | | | | X | | | | | |

A = nif1 replicative plasmid insertion;
B = nif2 replicative plasmid insertion;
C = nif4 replicative plasmid insertion;
D = anf4 replicative plasmid insertion;
E = anf5 replicative plasmid insertion;
F = nifH knockout;
G = GlnR site II inactivation;
H = GlnR site II duplication;
I = GlnR C25 truncation;
J = cueR knockout;
K = 77155(source)-nifB replicative plasmid insertion;
L = 103408 (source)-nifB replicative plasmid insertion;
M = hesA2 replicative plasmid insertion;
N = 77155(source)-katA replicative plasmid insertion;
O = 103408(source)-katA replicative plasmid insertion;
P = 77155(source)-sodA replicative plasmid insertion;
Q = 103408(source)-sodA replicative plasmid insertion;
R = 103408(source)-orf1 replicative plasmid insertion.
*Note:
Strain identifiers may further comprise an optional prefix, as shown in the table. For example, Parent Strain (PM)77155 with Edit Type C would be “(PE)77155-G128”, with the prefixes “PM” and “PE” for the parent and edited strains, respectively, being optional additional designations. Parental strain 77155 was sourced from New Zealand.

nif1

Introduction of a replicative plasmid into the host strain, the plasmid comprising: the entirety of the native sequence of the nif cluster from the nifB promoter to the terminator region downstream of nifV of Paenibacillus durus Strain 103408 (NRRL Deposit No. B-68113 deposited 7 Apr. 2022), the colE1 origin of replication, the ampR antibiotic resistance marker, the traJ origin of transfer, the emrC antibiotic marker, and the pUB origin of replication. The replicative plasmid introduced into the host cell resulted in duplication or higher-order replication of the inserted genes.

nif2

Introduction of a replicative plasmid comprising: a fragment of the native sequence of the nif cluster from the nifB promoter to the intergenic region downstream of orf1 of Paenibacillus durus Strain 103408 (NRRL Deposit No. B-68113 deposited 7 Apr. 2022), the colE1 origin of replication, the ampR antibiotic resistance marker, the traJ origin of transfer, the emrC antibiotic marker, and the pUB origin of replication. The replicative plasmid introduced into the host cell resulted in duplication or higher-order replication of the inserted genes.

nif4

Introduction of a replicative plasmid comprising: a fragment of the native sequence of the nif cluster from the nifB promoter to the intergenic region downstream of nifH of Paenibacillus durus Strain 103408 (NRRL Deposit No. B-68113 deposited 7 Apr. 2022), the colE1 origin of replication, the ampR antibiotic resistance marker, the traJ origin of transfer, the emrC antibiotic marker, and the pUB origin of replication. The replicative plasmid introduced into the host cell resulted in duplication or higher-order replication of the inserted genes.

anf4

Introduction of a replicative plasmid comprising: a fragment of the native sequence of the anf cluster from the anfH promoter to the intergenic region downstream of anfK of Paenibacillus durus Strain 103408 (NRRL Deposit No. B-68113 deposited 7 Apr. 2022), the colE1 origin of replication, the ampR antibiotic resistance marker, the traJ origin of transfer, the emrC antibiotic marker, and the pUB origin of replication. The replicative plasmid introduced into the host cell resulted in duplication or higher-order replication of the inserted genes.

anf5

Introduction of a replicative plasmid comprising: a fragment of the native sequence of the anf cluster from the anfH promoter to the intergenic region downstream of anfH of Paenibacillus durus Strain 103408 (NRRL Deposit No. B-68113 deposited 7 Apr. 2022), the colE1 origin of replication, the ampR antibiotic resistance marker, the traJ origin of transfer, the emrC antibiotic marker, and the pUB origin of replication. The replicative plasmid introduced into the host cell resulted in duplication or higher-order replication of the inserted genes.

nifH Knockout

Seamless (scarless) deletion of the entire nifH open reading frame while preserving all surrounding genetic material.

### GlnR Binding Site II Inactivation

Seamless (scarless) change of the last six nucleic acids of the GlnR binding site II sequence to the non GlnR binding sequence “ATCGAT”.

### GlnR Binding Site II Duplication

Seamless (scarless) replacement of the native GlnR binding site I sequence with an additional copy of the native GlnR binding site II sequence.

### GlnR C25 Truncation

Seamless (scarless) deletion of the nucleic acids comprising the coding sequence for the last 25 amino acids of the glnR open reading frame, with preservation of the native stop codon and all surrounding sequence.

### CueR Knockout

Seamless (scarless) deletion of the entire cueR open reading frame while preserving all surrounding genetic material.

### Parent and Control Strains

Paenibacillus polymyxa Strain 77155 (NRRL Deposit No. B-68191 deposited 18 Aug. 2022), Strain 77155-G3 comprising a genomic GlnR Binding Site II inactivation mutation, Strain 77155-G14 comprising a genomic NifH knockout, Strain 77155-G27 comprising a genomic GlnR Binding Site II duplication, Strain 77155-G46 comprising a genomic GlnR C25 truncation, and Strain 77155-G66 comprising a genomic CueR knockout were used as parent strains for editing as described herein.

The appropriate control for assays was the parental strain with the pBACOM empty vector, and not the wild type (WT) parent strain. Because the edited strains were selected with antibiotics and the WT strains didn't comprise an antibiotic selectable marker, and the antibiotic suppresses all pathways, the GE strains grown in the presence of the antibiotic would have looked artificially low compared to the WT. Control strains included 77155-G188 (also called 77155-MLS-R of 77155-G201, is the Wild Type Strain 77155 transformed with a pBACOM empty vector), 77155-G208 (also called 77155-G202, is Strain 77155-G3 comprising a genomic GlnR Binding Site II inactivation mutation, transformed with a pBACOM empty vector), Strain 77155-G210 (Strain 77155-G9 comprising a genomic GlnR Binding Site II duplication & inactivation mutation, transformed with a pBACOM empty vector) Strain 77155-G203 (Strain 77155-G14 comprising a genomic NifH knockout, transformed with a pBACOM empty vector), Strain 77155-G204 (Strain 77155-G27 comprising a genomic GlnR C25 truncation, transformed with a pBACOM empty vector), Strain 77155-G205 (Strain 77155-G46 comprising a genomic GlnR C25 truncation, transformed with a pBACOM empty vector), and Strain 77155-G206 (Strain 77155-G66 comprising a genomic CueR knockout, transformed with a pBACOM empty vector).

### Replication of Some or all of the Nif Gene Cluster

### On a Plasmid

The most straightforward and rapid way to test if increasing the number of copies of the nif cluster would result in an increase in nitrogen fixation would be using a replicative plasmid.

The nif gene cluster will be synthesized or amplified via PCR, cloned into a mobilizable replicative plasmid, then transformed the target strain by conjugation. The resulting transconjugants will be screened for ARA activity. However, there are some complicating considerations.

An exact copy of the target strain's existing nif cluster would not be used, as this would likely cause recombination between the copy on the plasmid and the native sequence. There are several approaches to preventing this, and each would reveal something slightly different.

One approach would be to recalibrate the codon usage of the sequence in silico, then have this new “synthetic” sequence synthesized. The synthetic nif cluster would express the exact same suite of proteins, but would have a completely different genomic sequence, preventing recombination. This approach would answer the question of whether the presence of additional copies of the nif cluster is sufficient to increase nitrogen fixation.

Another approach would be to mine other genomic sequences for a nif operon that is similar to the native cluster of the target strain, but different enough that recombination is unlikely to occur. This cluster could be quickly amplified from the source strain's genomic DNA for cloning into a plasmid. This approach would be more rapid and less expensive than synthesizing the entire biosynthetic gene cluster. The results of this experiment would tell us if having a copy of a different nif operon increases nitrogen fixation-if it is beneficial to have different versions of the cluster present.

A third option is to take a Nitrogen-fixing Paenibacillus and remove the native cluster. That would give a clean background to work in and eliminate a lot of chances for recombination.

As the size of plasmids increase conjugation efficiencies decrease. The Paenibacillus polymyxa nif cluster is approximately 10.7 kb, so plasmids would be fairly large. It would be important to use parent strains with high conjugation efficiencies.

Additionally, the copy number of the plasmid is significant. High copy number plasmids expressing the nif cluster would likely place a high metabolic burden of the recipient strain and could potentially be toxic. It is important to test several Gram-positive replicative plasmid backbones with different relative copy numbers to determine the ideal copy number for seeing an increase in nitrogen fixation

### Experimental Plan:

Select a parent strain based on high transformation efficiency by conjugation, antibiotic susceptibility, and compatibility with the ARA assay
Design a synthetic, codon optimized nif cluster and order from a DNA synthesis vendor.
Contemporaneously, mine the genomic library for Paenibacillus isolates with proven nitrogen fixation activity, where the nif clusters are similar in their protein sequence to the parent strain, but sufficiently different in their genomic sequences (less than 90% similarity). Design and order plasmids for amplification.
Identify and obtain a suite of Gram-positive vector backbones with a variety of copy number levels. Ensure that they are mobilizable by conjugation
Clone the synthesized, and/or native nif clusters into the selected plasmids
Mobilize the plasmids into the target strain via conjugation.
Assay the ability of recovered transconjugants to fix nitrogen using ARA

### Chromosomal Integration

Chromosomal integration would be an important step for bringing the lessons learned in plasmid-based studies towards development of an intrageneric gene edited project. It would also be a potential next step if copy number issues stymie a plasmid-based approach. An integrative approach, particularly using transposon integration or Cre-lox integration could bypass issues associated with having too many copies of the gene cluster.

An integration approach would have many of the same considerations as the plasmid approach in terms of difficulty of conjugation scaling with the size of the plasmid, and concerns over unwanted homologous recombination with extra copies of the parent strain's native cluster.

One approach would be to utilize Homologous Recombination to simultaneously attempt to insert in the entire cluster, half of the cluster, and a third of the cluster, a quarter of the cluster, and/or gene by gene and proceed with inserting remaining segments as needed depending on which is successful. In an approach like this there could be the first insertions marked with an antibiotic marker, to be replaced by subsequent insertions with a different antibiotic marker. The approach could be made scarless just having the final insertion be unmarked, or by leaving the final insertion marked and return later to scarlessly remove the marker. Since this is a targeted approach, a neutral site for integration would be useful.

Integrating an extra copy of the nif cluster using a random insertion transposon is another way to get integration. The synthetic or natural nif cluster would be cloned into a mobilizable suicide delivery transposon, or mariner transposon vector and conjugated into the selected parent strain. The transposon comprising the nif cluster and a selectable marker would randomly integrate upon mobilization, with each recovered transconjugant having integrated in a different location. The transconjugants could then be screened to see which insertion event yields the best activity. If using a mariner transposon vector (which mobilizes as a replicative vector before expressing the transposase) it may be possible to yield multiple insertions.

A significant drop in efficiency in transposons vs replicative vectors is sometimes observed, so conjugation efficiency is of extra concern. It may be helpful to choose a strain that has known compatibility with mariner transposon vectors. The issue with increasing plasmids sizes would also apply to this approach, particularly considering the size of the mariner transposon vectors (over 10 kb).

Once activity is confirmed, transconjugants could be made intrageneric by removing the transposon flanking sequences (these constitute intergeneric sequence) using scarless homologous recombination.

A benefit of transposon integration over homologous recombination is that bespoke vectors would not need to be made for each parent. The same integration plasmid could be used to deliver the same sequence to multiple different targets. The downside is that this delivery would never be targeted.

Alternatively, Cre-lox is a tool used for DNA recombination that can be used to insert large DNA sequences into a targeted genomic region. First, a “landing pad” comprising two specific sequences (lox sequences) that can be targeted for recombination by the Cre protein is integrated into the targeted location using homologous recombination. The landing pad can be marked with an antibiotic cassette. Large sequences can be swapped with the AB marker between the lox sites by introducing a plasmid in which the gene cluster is flanked by lox sites and contains a Cre expression cassette. When the plasmid is introduced, Cre is expressed, swapping the locations of the antibiotic marker and the sequence to be mobilized. An additional antibiotic marker can also be used to assist with selection of proper edits. The final sequence will contain intergeneric DNA in the form of the flanking lox sites and any remaining antibiotic marker. These elements could be later removed using homologous recombination.

A benefit if using a Cre-lox approach is that once a mutant with a “landing pad” is established, multiple different “cargo” sequences can be mobilized rapidly. This would allow for the same plasmid to be delivered to multiple landing pad containing strains, and would simplify cloning by removing the need for homologous arms for each tested integration. This provides the advantages of using a transposon, with the added ability to target the delivery.

nif Cluster Partial or Complete Duplication, Replacement and/or Addition with a Near Relative of Paenibacillus nif Gene Cluster

In one example, the nif gene cluster from a nitrogen fixing bacteria is introduced into a nitrogen fixing Paenibacillus bacterium expressed in the chromosome or on a plasmid downstream or its native promoter and/or downstream of the target Paenibacillus gene cluster promoter. Introducing the additional nitrogen fixation gene cluster is expected to increase organismal overall nitrogen fixation by providing additional nitrogenase and cofactor support enzyme(s) necessary for fixation.

In another example the native nitrogen fixation gene cluster of Paenibacillus is removed and an exogenous nitrogen fixation gene cluster including, but not limited to subgroup I or II Paenibacillus is expressed in the chromosome and/or on a plasmid utilizing the native promoter or the promoter from the target Paenibacillus. The replacement of the nitrogen fixation gene cluster with an exogenous nitrogen fixation gene cluster is expected to attenuate the conditions in which nitrogen fixation occurs including increasing or decreasing oxygen and/or nitrogen sensitivity.

In another example, a nitrogen fixation gene cluster is expressed on a chromosome or on a plasmid in Paenibacillus strain which did not contain a functional nitrogen fixation gene cluster. The addition of the nitrogen fixation gene cluster into a strain which does not natively contain the nitrogen fixation cluster will confer nitrogen fixation activity to the new microorganism.

A nifA Gene from a Near Relative of Paenibacillus (e.g., Frankia) is Introduced into the Paenibacillus Bacterium to Activate the nfix Gene Cluster and/or to Bind to a nifL Like Negative Nitrogen Fixation Regulator

In one example the nifA gene from a non-Paenibacillus nitrogen fixing bacteria is inserted into a nitrogen fixing Paenibacillus, previously engineered or unengineered, chromosome, or expressed on a plasmid, downstream of its native promoter, a promoter from the target Paenibacillus, and/or the promoter sequence found upstream of the nitrogen fixation gene cluster in the target Paenibacillus. Introducing the nifA gene is expected to activate nitrogen fixation either by directly acting on the native nitrogen fixation biosynthetic gene cluster and/or binding to nifL like negative regulators of nitrogen fixation. The nitrogen fixing capabilities of these nifA engineered strains are measured using standard assays known in the art.

In another example, the inserted nifA protein includes a protein tag, e.g. His-6, FLAG, and this tag is used to isolate the nifA protein from bacterial lysate to determine if and which, through downstream protein identification studies, native proteins which bind to nifA.

anf and/or vnf Gene(s) May be Duplicated; anf and/or vnf Gene(s) May be Upregulated; anf and/or vnf Gene(s) May be Introduced into a Strain of Subgroup I, Such as Paenibacillus polymyxa; the anf/vnf Cluster Less Sensitive to O2, and Introducing into Polymyxa May Make it Less Sensitive to Oxygen, or Function in the Absence or Limitation of a Rare Element;

In one example all or pieces of the anf and/or vnf gene clusters, determined either bioinformatically or by other experimental methods, are inserted into the chromosome, and/or on a plasmid, of an engineered or unengineered nitrogen fixing Paenibacillus which either contains a native copy of the anf and/or vnf gene clusters or lacks said genes. The inserted anf or vnf gene clusters are under the regulation of their native promoter sequences, constitutive promoters, and/or the promoter sequences which regulate the canonical Paenibacillus nitrogen fixation gene cluster. The introduced gene clusters can all be expressed at higher than basal levels or differentially expressed depending on the promoters used and the method of insertion, i.e., chromosomal integration or plasmid expression, into the target Paenibacillus.

In another example all or part of the anf and/or vnf gene clusters are inserted into an nitrogen fixing Paenibacillus polymxa from Subgroup I as described above. This engineering creates a modified Paenibacillus polymyxa nitrogen fixing bacterium which is less sensitive to downregulation of nitrogen fixing by oxygen and/or capable of fixing nitrogen even in the absence or low concentration of essential nitrogenase cofactors e.g., iron, molybdenum, vanadium.

The nifB Genes in a Subgroup II Strain May be Streamlined; Duplication of Some or all of the Cluster within the Microbe's Genome May be Effected, for Example by Providing Multiple Copies of nifB, as the Instigator of the Cluster; the nifB Gene of Polymyxa is Longer than Other nifBs;

In one example each of the distinct nifB coding sequences, along with its respective native regulatory sequences, in the nitrogen fixing Paenibacillus subgroup II strain is moved from its native location in the genome such that it is positioned upstream of the canonical nitrogen fixation gene cluster. Alternatively, all naturally occurring nifB genes are unaltered in Paenibacillus subgroup II strain, and the copies of the nifB coding sequences are inserted upstream of the canonical nitrogen fixation gene cluster under the regulation of either their native promoter sequences or the canonical nitrogen fixation gene cluster regulation machinery in its unengineered or engineered form, e.g., Site II duplication, Site II inactivation, and/or GlnR Site II inactivation and duplication.

In another example, the nifB gene and unengineered or engineered regulatory sequences from a nitrogen fixing Paenibacillus polymyxa are inserted in front of the distinct Subgroup II nifB genes. These engineered strains will have greater nitrogen fixing capabilities as measured using standard assays known in the art.

Gene Expression Across nifH, anfH, and/or vnfH May be Synchronized in a Subgroup II Strain with the Same Promoter and nifB Gene.

In one example, expression of multiple nitrogenase clusters (nif and anf or vnf) present in Paenibacillus Subgroup II sp. genomes will be synchronized by substitution of native promoter elements upstream from anfH/vinfH for the canonical nitrogen fixation gene cluster regulation machinery in its unengineered or engineered form (e.g., Site II duplication, Site II inactivation, and/or GlnR Site II inactivation and duplication) and nifB open reading frame. The canonical nitrogen fixation gene cluster regulation machinery in its unengineered or engineered form (e.g., Site II duplication, Site II inactivation, and/or GlnR Site II inactivation and duplication) and nifB coding sequence will be substituted for anfH/vnfH promoters via homologous recombination of cloned regulatory and coding sequences.

In another example, the anf/vnf nitrogenase clusters could be expressed from a plasmid controlled by the canonical nitrogen fixation gene cluster regulation machinery in its unengineered or engineered form (e.g., Site II duplication, Site II inactivation, and/or GlnR Site II inactivation and duplication). To accomplish this, the genes anfHDGK or vnfHDGKEN would be cloned into a plasmid downstream from the canonical nitrogen fixation gene cluster regulation machinery in its unengineered or engineered form (e.g., Site II duplication, Site II inactivation, and/or GlnR Site II inactivation and duplication) and nifB coding sequence which would allow for synchronized expression of multiple nitrogenase complexes that utilize different metal cofactors.

Nitrogenase Function can be Protected from Oxidative Stress by Increasing Expression of any Combination of Catalase (katA, katE, katX, and Related Homologs), Superoxide Dismutase (sodA, sodC, sodF, and Related Homologs), Thioredoxin (trxA, trxB, and Related Homologs), Peroxidase Including Alkylhydroperoxidase C (ahpC and Related Homologs).

In one example, expression of chromosomally-encoded oxidative stress protective enzymes (any combination of catalase (katA, katE, katX, and related homologs), superoxide dismutase (sodA, sodC, sodF, and related homologs), thioredoxin (trxA, trxB, and related homologs), peroxidase including alkylhydroperoxidase C (ahpC and related homologs)) could be increased by substitution of native promoters for those known to exhibit high activity (e.g., PaprE).

In another example, expression of chromosomally-encoded oxidative stress protective enzymes (any combination of catalase (katA, katE, katX, and related homologs), superoxide dismutase (sodA, sodC, sodF, and related homologs), thioredoxin (trxA, trxB, and related homologs), peroxidase including alkylhydroperoxidase C (ahpC and related homologs)) could be synchronized with nitrogenase expression by substitution of native promoters for canonical nitrogen fixation gene cluster regulation machinery in its unengineered or engineered form (e.g., Site II duplication, Site II inactivation, and/or GlnR Site II inactivation and duplication).

In another example, oxidative stress protective enzymes (any combination of catalase (katA, katE, katX, and related homologs), superoxide dismutase (sodA, sodC, sodF, and related homologs), thioredoxin (trxA, trxB, and related homologs), peroxidase including alkylhydroperoxidase C (ahpC and related homologs)) could be expressed from a plasmid under the control of an inducible promoter or via canonical nitrogen fixation gene cluster regulation machinery in its unengineered or engineered form (e.g., Site II duplication, Site II inactivation, and/or GlnR Site II inactivation and duplication).

### Example 3: Cloning

Cloning vectors were assembled by introducing an editing cassette (described above) into the pMMmob backbone. pMMmob [oriBsTs traJ ecol1 mls amp] is a derivative of the plasmid pMiniMAD2 obtained from the Bacillus genetic stock center. pMMmob is digested with the restriction enzymes BamH1 and EcoR1, run on a 10% agarose gel, and purified.

Upstream and downstream homology regions are amplified from genomic DNA extracts via PCR using proof reading polymerase, and primers designed to append flanking sequences for subsequent Gibson assembly. For constructs in which a sequence is added between the flanking homology arms, the sequence introduction is achieved by inclusion in the primer flanking sequences. The PCR products were run on a 10% agarose gel and purified.

The backbones and inserts were combined at a 1:3 backbone-to-insert molar ratio, combined with Gibson assembly reagent, and incubated at 50° C. for 60 minutes for plasmid assembly. The Gibson assembly mixtures were subsequently transformed into chemically competent DH5a E. coli. Transformants were recovered on LB+100 ug/uL Ampicillin plates.

Proper assembly of the plasmids was confirmed by restriction digest analysis and PCR of the insert region. The editing cassette was sequenced using Sanger sequencing to confirm the absence of off-target mutations.

Confirmed plasmids were extracted from overnight DH5α cultures and transformed into electrocompetent BW29472 E. coli via electroporation and recovered onto LB+100 ug/uL Ampicillin+300 μM diaminopimelic acid plates for conjugation into the host strains.

### Example 4: Gene Editing in Paenibacillus spp.

### Scarless Homologous Recombination

This is a general protocol for gene editing in Paenibacillus using a temperature sensitive scarless homologous recombination plasmid and is used for edits described herein. This protocol was developed for editing and may be broadly applicable to Paenibacillus isolates. This protocol requires prior assembly of one or more editing vectors designed for the desired edits using pMMmob backbone hosted in an E. coli donor strain and one or more Paenibacillus recipient strains with confirmed susceptibility to the relevant antibiotic resistance marker.

### Conjugation

The desired recipient strains are grown overnight in appropriate growth medium. Donor strains are grown overnight in appropriate growth medium supplemented with the relevant antibiotic marker for maintenance of the mobilizable plasmid. Aliquots of the overnight culture are washed, combined, and plated onto appropriate agar medium for growth of both strains. These plates are incubated overnight at the permissive temperature for plasmid replication in the recipient strain.

The mating mixtures are recovered, washed, and replated onto agar plates supplemented with the appropriate antibiotic marker for selection of transconjugant recipient strains. The plates are incubated overnight at the permissive temperature for plasmid replication until the appearance of transconjugant colonies.

### Integration

Transconjugant colonies are grown in liquid culture in the presence of the selective marker at the permissive temperature for plasmid replication overnight. Dilutions of the liquid culture are plated onto agar plates supplemented with the selective antibiotic and incubated overnight at the restrictive temperature for plasmid replication. Colonies recovered under these conditions are assumed to have integrated the editing plasmid by homologous recombination.

### Excision

Integrated colonies are inoculated into liquid culture, grown to turbidity at the permissive temperature for plasmid replication, then subcultured into fresh medium lacking the antibiotic and grown overnight again at permissive temperature. This serial subculturing is repeated 2-3 more times, and dilutions of the final subculture are plated onto agar plates lacking the antibiotic.

Recovered colonies are assayed for loss of the plasmid by replating onto medium containing and lacking the antibiotic. Colonies that grow in the absence of the antibiotic but not in the presence of the antibiotic are confirmed to have excised and lost the plasmid and were identified as putative edited strains.

### Confirmation

Putative edited strains are screened to determine if the edit was successfully delivered, or if the strain reverted to wildtype, by amplifying the editing region via polymerase chain reaction (PCR). PCR products are analyzed by gel electrophoresis and Sanger sequencing to confirm the appropriate product size and sequence for the edit. Colonies confirmed to have the edit successfully delivered are checked via Sanger sequencing to confirm no off-target mutations were added to the edit region, and for lack of growth on medium containing the antibiotic to confirm no presence of plasmid backbone.

### Other Methods

Alternatively, any other method known in the art may be employed to effect any one or more of the polynucleotide edits described herein, for example but not limited to: targeting and/or homing nucleases, restriction endonucleases, zinc finger nucleases, meganucleases, Cas endonucleases, TAL effector nucleases, guided nucleases, random site mutations, blind editing, chemical mutagenesis, or radiation mutagenesis. Generally, a double-strand break is created at or near the target site to be edited, which is repaired by intracellular processes such as non-homologous end joining, homologous recombination, or homology-directed repair. In bacteria, Cas endonucleases may be used to counter-select against unedited strains after introduction of a DNA repair template which is incorporated into the chromosome by homologous recombination. The targeting of the Cas endonuclease to the wild-type unedited strain allows for selection of only cells that have incorporated the desired modification. The net effect can be any one or more of the following: insertion of at least one nucleotide, deletion of at least one nucleotide, replacement of at least one nucleotide, chemical alteration of at least one nucleotide. For the purposes of the edits of Paenibacillus strains described herein, any technique that is desired by the practitioner may be used to achieve the end result.

### Example 5: Microbe Identification and Storage

Sequencing preparation for microbe identification, and long-term storage, was performed by the following method:

### Day 1:

Use a 10 uL sterile tip to transfer a colony from a plate to a flask containing an appropriate liquid growth medium. Place the isolates on a shaker at room temperature and incubate for 2 days.

### Day 3:

Tubes may be turbid after being on the shaker for 2 days. All samples are analyzed by PCR. Vortex each tube, collect a 50 uL sample from each vortexed tube, and dispense in a 96-well plate. Using a multichannel pipette, dispense 15 uL of the 50 uL samples into a new 96-well plate. The 96-well plate containing 35 uL of each sample will be used for phenotyping, and the 96-well plate containing 15 uL of each sample will be used for PCR analysis. 27F/1492R primers are generally used for 16S PCR analysis, as they yield better results than PB36/38. Appropriate negative controls should be included with the plate and analyzed by PCR. The plate will be analyzed by PCR using an Eppendorf thermocycler. Once the PCR is finished, run a gel using standard gel electrophoresis techniques. This is important because most isolates are grown enough where they should ideally be put into long-term storage on day 3. The PCR and gel electrophoresis analysis are used to confirm that the isolates contain bacteria, rather than other microbes. For isolates that do not pass PCR or have clear broth, vortex tubes and use a loop to streak out onto a petri plate. Check after several days to see if anything grows, or if the tube is contaminated. For isolates that pass PCR, dispense 600 uL of 50% glycerol into a 2 ml screw cap tubes and add 1200 uL of the bacterial culture, such that the broth is stored in 20% glycerol. Store the glycerol stock at −80° C. and record an image of the gel of the PCR samples.

### Day 4:

Check the petri plates of the streaked isolates that failed PCR for growth. (During this time, the 2 ml broth tubes will remain on the shaker.) Once there is growth on the plate and the colonies appear to have been successfully isolated, dispense 600 uL of the broth-glycerol mixture in the small tube, and put both tubes in their respective −80 boxes. It is possible for isolates to fail the PCR check because of any of the following reasons: the primers may not work on all bacteria, the isolate is actually a fungus, the isolate is very adherent and therefore does not homogenize in the broth, the isolate produces too much of a interfering compound such as EPS and therefore needs dilution prior to PCR set-up, or the isolate is a slow grower. Over the next few days, continue checking the plate to confirm that only a single bacterial species was isolated. If contamination is observed, prepare a new isolate. Viability of the prepared glycerol stocks should be verified.

### Example 6: Formulation of Microbes

Microbes identified according to the previous examples may be formulated with additional components for application via methods such as, but not be limited to: seed treatment, root drench, root wash, seedling soak, foliar application, soil inocula, in-furrow application, sidedress application, soil pre-treatment, wound inoculation, drip tape irrigation, vector-mediation via a pollinator, injection, osmopriming, hydroponics, aquaponics, aeroponics. The formulation comprising the microbes are prepared for agricultural application as a liquid, a solid, or a gas formulation. Application to the plant is achieved, for example, as a powder for surface deposition onto plant leaves, as a spray to the whole plant or selected plant element, as part of a drip to the soil or the roots, or as a coating onto the plant element prior to planting. Such examples are meant to be illustrative and not limiting to the scope of the invention.

Media components for an exemplary microbe preparation are shown below in Table 2. Add all contents with 50% of the final volume of water needed, and stir the solution at an elevated temperature until dissolved. After all contents have been dissolved, use sterile RO water to bring the solution to the final desired volume. Field trial preparations are typically performed using the 4x formulation.

TABLE 2
Exemplary media components and concentrations for microbe formulation

| | | | | fill vol. | | | | fill vol. |
|---|---|---|---|---|---|---|---|---|
| | 0.9X | fill vol. | 1.1x | below | 2x | fill vol. | 4x | below |
| | 1 ml | below | 30 ml | 30 | 1 ml | below | 1 ml | 10 |
| Xanthan Gum (mg) | 1.8 | 0 | 2.2 | 66 | 4 | 0 | 8 | 80 |
| Trehalose (mg) | 45.3 | 0 | 55 | 1650 | 100 | 0 | 200 | 2000 |
| Isomalt (mg) | 22.65 | 0 | 27.5 | 825 | 50 | 0 | 100 | 1000 |

Exemplary media components for microbe formulation

| Component | CAS# |
|---|---|
| Sucrose | 57-50-1 |
| Proflo | 68308-87-2 |
| Yeast extract | 8013-01-2 |
| Tryptone | 91079-40-2 |
| MgSO4•7H2O | 10034-99-8 |
| KH2PO4 | 7778-77-0 |
| K2HPO4 | 7758-11-04 |
| NaNH4HPO4•4H2O | 7783-13-3 |
| CaCl2 | 10043-52-4 |
| MnSO4•1H2O | 10034-96-5 |
| ZnSO4•7H2O | 7446-20-0 |
| CuSO4•5H2O | 7758-99-8 |
| Na2MoO4*2H20 | 10102-40-6 |
| FeSO4*7H20 | 7782-63-0 |
| Tryptone | 91079-40-2 |
| Soy peptone | 91079-46-8 |
| NaCl | 7647-14-5 |
| K2HPO4 | 7758-11-4 |
| Glucose | 50-99-7 |
| Trehalose | 6138-23-4 |
| Isomalt | 64519-82-0 |
| Xantham gum | 11138-66-2 |

The procedure to mix TIX formulation is as follows: Measure all dry ingredients into a 50 ml tube. Vortex the ingredients well to ensure xanthan gum is “separated” through the other carbon sources. Add about half of total sterile RO water to the mix, vortex. Use the long end of an L-spreader to break up chunks as much as you can. Heat some sterile RO water in the microwave to warm water bath temperature (45-50° C.). Add the remaining sterile RO water to the mix, vortex. Repeat step 4 and vortex as needed until you have a clear solution with no lumps. Spin down the bubbles created in the process of mixing by using a centrifuge for 5-10 seconds on “fast spin”. Remember to have a balance to counter the formulation (TIX) tube. Allow formulation to cool to room temperature. 1. Mix in the microbial consortia. Vortex to ensure homogeneity. It is ideal to add microbes at a concentration of 10\hat{ }9 CFU/ml to the formulation.

Apply the formulation to the plant or plant element for testing in field trial.

### Example 7: Application of Microbes to Plant Elements and Cultivation Thereof

A microbial composition (comprising one or more isolated microbes of a single strain, a consortium, a community, a combination, or any combination of the preceding) is prepared according to the previous Examples. The microbial composition comprises one or more microbes, optionally in combination with one or more additional microbes disclosed herein.

### Microbial Compositions for Application

In some methods, the microbial composition is dried and applied directly to a plant element.

In some methods, the microbial composition is suspended in a liquid formulation for application to a plant element.

In some methods, the microbial composition is combined with another composition, such as but not limited to: a carrier, a wetting agent, a stabilizer, a salt. In some methods, the other composition comprises a molecule that introduces additional agriculturally-beneficial outcomes to the plant to which the microbial composition is applied. The other composition includes, for example but not limited to: an herbicide, a fungicide, a bactericide, a pesticide, an insecticide, a nematicide, a biostimulant.

### Application Types

The microbial composition is applied to a plant element, at a time during development appropriate to the desired outcome, for example: in a formulation of a pre-planting soil drench/in-furrow application; as a seed or other reproductive element treatment; as a post-planting reproductive element application; as an in-furrow, drip, or drench application after planting; as a direct application to a plant element (e.g., root, leaf, stem); as an application to a harvested plant element (e.g., a fruit or a grain). Combinations of application types are also tested.

### Application Methods

The microbial composition is applied to (inoculating) a plant or plant element or plant product (pre-planting, post planting, pre-harvest, or post-harvest). This can be accomplished, for example, by applying the agricultural composition to a hopper or spreader or tank, which contains the microbial composition and which is configured to broadcast the same.

A seed coating of the microbial composition is applied to one or more seeds of a crop plant. Upon applying the isolated microbe as a seed coating, the seed is planted and cultivated according to practices established for that crop.

Alternatively, the microbial composition is applied to the soil for the benefit of a plant existing in that soil. Methods of soil application include in-furrow treatment, drench, and drip applications.

Alternatively, the microbial composition is applied to the surface of a plant or plant part after germination.

Alternatively, the microbial composition is applied to material obtained from the plant after harvest.

A control plot of plants, which did not have the isolated microbe applied, are also planted. Plants associated with the microbial composition exhibit improved characteristics of interest.

Application methods may be performed according to any protocol known in the art.

Plant elements, plants, or growth medium (e.g., soil) may further be inoculated with a disease or pest, according to the purpose of the test.

An exemplary, non-limiting protocol for drenching tomato plants is given below:
1. Ten days after planting carefully separate plants out into 6 reps for each treatment. Plants are delicate and leaves can tear easily. Ensure that the size and overall appearance of plants is as uniform as possible (The purpose of thinning is continuing with an homogenous plant population). Transplant if there are not enough plants per reps. See step 3 for guidelines on transplanting.
2. Begin thinning pots down to one plant per pot. Remove the smaller plant, one that is unhealthy or deformed in some way. If there are 2 or more healthy plants per pot, the extras can be transplanted into another pot. Use leftover soil prepped from initial planting or from pots where seeds did not germinate.
3. To transplant: If some pots didn't germinate, they can be filled with a plant from another container. To do this simply scoop out the extra plant (trying to scoop out as much root mass as possible without disturbing the other plant) with a scoopula and place into a hole made in the empty pot. Firm the soil around the plant with slight finger pressure.
4. Space out the pots into 6 pot lines (1 line of pots per treatment), will take 4 RL98 trays. Once done, have a look at all the treatments and consider making some pot switches to ensure some treatments don't have all large plants and others have all large plants.
5. Change gloves if necessary. Label each pot with your pre-prepared Avery Labels. Treatments should be labeled into rows of 6 replicates i.e., 1-1, 1-2, 1-3 to 1-6, etc. Makes it easier to find all replicates for each treatment
6. Two weeks after planting (roughly 4 days after thinning and labeling), obtain treatments from the Microbiology team; set on the table with trays of prepped plants. Gather combitips, repeater, and RO water. (note: Plants should be watered lightly the day of treatment)
7. Mix microbial solution by inverting tube/container (microbial treatment) 2-3 times or give a light shake. Set combitip to dispense 2 ml. Collect treatment fluid into combitip, dispense first step back into the tube. Ensure the treatment you have corresponds to the row of plants to be treated. Once confirmed, gently dispense 2 ml of treatment onto the surface soil of each pot, close to the stem but avoid direct contact with the stem and leaves.
8. Dispose of combitip and repeat step 6 for all treatments. For the inoculated control (IC or InoCon) and untreated control (UTC), apply RO water in place of a treatment. Once all treatments have been applied, place plants back into growth chamber for (optional inoculation), growth, evaluation.
   Visualization of Microbes Associated with Plant Elements

Individual microbes can be tagged with a fluorescent protein according to methods known in the art. Microscopic image analysis demonstrated that the microbes disclosed herein were found associated with various plant tissues.

### Example 8: In Vitro Testing

Wild Type and Genome-edited strains were assessed for root colonization, acetylene reduction activity, biofilm formation, turbidity (OD at 600 nm), oxygen tolerance, and gene expression. Unless otherwise specified, protocols are conducted using methods known in the art.

ARA with GC-FID for Gram Positive Strains

Ensure all equipment and materials are sterilized. Wrap sealing equipment containers in foil prior to autoclaving so that they can be unwrapped in the Anaerobic chamber pass box and enter the Anaerobic chamber sterile. Seal vial openings with foil prior to sterilizing. Loose ‘seals’ are required to allow gas exchange in the pass box.
1. Streak isolates from −80 C and incubate at 30 C or 25 C until colonies are observed.
2. Spread one plate/isolate and incubate at 25 C or 30 C until a lawn is observed.
3. Harvest plates and OD600 balance each isolate to approx. 0.3 to normalize the inoculant.
4. Prepare the Anaerobic chamber by cleaning the surfaces and passing the sealing equipment through—ensure containers allow for gas exchange.
5. Add 30 mL NF11/vial-3 reps/isolate.
6. Add 150 uL inoculant/vial which was balanced to an OD600 of 0.3 using sterile water.
7. Pass vials through Anaerobic chamber and seal under anaerobic conditions-include an empty vial (with foil ‘cap’) to add an anaerobic indicator for QC purposes.
8. Place vials in 30 C, 200 rpm for 5 hours.
9. After 5 hours, working in the fume hood, remove 10% (4 mL) from the headspace of each vial and replace with the same volume of acetylene gas. NOTE: Acetylene gas is highly reactive and explosive thus the bag must be kept in the fume hood while working.
10. Incubate at 30 C, 200 rpm for 48 hrs.
11. At 48 hours (or other known timepoint), take 1 mL headspace sample and place into a GC collection tube.
12. Run samples in GC using instrument method for ethylene analysis ‘split 4’ which measures acetylene peak and ethylene people
13. Amount of gas is quantified by peak area.
14. Take OD600 readings of 200 ul of the culture and TVCs of culture.
15. Analyze ethylene gas as a percentage conversion of acetylene to ethylene. This produces an estimate of total conversion.

The volume of gas produced (ethylene) can either be quantified using calibration points in Chromeleon or by calculation from the % peak area. Acetylene+Ethylene peak area % must =100% for this. From the knows amount of Acetylene added, the ethylene produced can be determined in mL. 1M of gas=24 dm3 or 24,000 ml. Therefore 1 mM of gas=24 ml.

To calculate how many mM ethylene produced, divide amount by 24: mM ET=ml/24

To calculate RATE: mM per hour per CFU, you need to calculate mM as described above, and need to know how much of the headspace you sampled (if using calibration calculation e.g., 1 mL of headspace sampled has x mM gas but there is 6 mL headspace total so total ethylene produced=6x mM). If calculating using peak area % only the above step is not necessary, just need to know how much acetylene you added. Need to know the number of hours of incubation with Acetylene. Need to do TVCs to calculate CFU/ml then multiple your CFU value by the number of ml cultured e.g., 4 mL (gneg) or 30 mL (gpos).

Rate=total ethylene mM/(time (h)×total CFU)

ARA with Oxygen Tolerance Testing Protocol

Ensure all equipment and materials are sterilized. Wrap sealing equipment containers in foil prior to autoclaving so that they can be unwrapped in the Anaerobic chamber pass box and enter the Anaerobic chamber sterile. Seal vial openings with foil prior to sterilizing. Loose ‘seals’ are required to allow gas exchange in the pass box.
1. Streak isolates from −80 C and incubate at 30 C or 25 C until colonies are observed.
2. Spread one plate/isolate and incubate at 25 C or 30 C until a lawn is observed.
3. Harvest plates and OD600 balance each isolate to approx. 0.3 to normalize the inoculant.
4. Prepare the Anaerobic chamber by cleaning the surfaces and passing the sealing equipment through-ensure containers allow for gas exchange.
5. Take NF11 media into the Anaerobic chamber after cleaning. Add agar at 20 g/L to NF11 and place on hot plate. Briefly bring to boil to melt the agar. After melting agar, pour 30 mL of the e warmed agar into 70 mL on their sides to maximize surface area to produce slants.
6. Add 150 uL inoculant/vial which was balanced to an OD600 of 0.3 using sterile water. Do so trying to maximize the surface area exposed to the inoculate.
7. Pass vials through Anaerobic chamber and seal under anaerobic conditions-include an empty vial (with foil ‘cap’) to add an anaerobic indicator for QC purposes.
8. To adjust oxygen levels, after sealing the vial, take a thin needle syringe and remove the portion of anaerobic air from the via which will be replaced with 100% pure medical grade oxygen.
9. The assay has been run at various oxygen conditions from 0% oxygen to 22% oxygen and can be increased to much higher oxygen conditions due to manual addition of oxygen. For example, to achieve 5% oxygen, remove 2.2 mL anaerobic gas by hand and add 2 mL of 100% pure oxygen back at this condition.
10. Place vials in 30 C incubator for 5 hours.
11. After 5 hours, working in the fume hood, remove 10% (4 mL) from the headspace of each vial and replace with the same volume of acetylene gas. NOTE: Acetylene gas is highly reactive and explosive thus the bag must be kept in the fume hood while working.
12. Incubate at 30 C, 200 rpm for 48 hrs.
13. At 48 hours (or other known timepoint), take 1 mL headspace sample and place into a GC collection tube.
14. Run samples in GC using instrument method for ethylene analysis ‘split 4’ which measures acetylene peak and ethylene people
15. Amount of gas is quantified by peak area.
16. Analyze ethylene gas as a percentage conversion of acetylene to ethylene. This produces an estimate of total conversion.

Volume of gas produced (ethylene) can either be quantified using calibration points in Chromeleon or by calculation from the % peak area. Acetylene+Ethylene peak area % must=100% for this. From the knows amount of Acetylene added, the ethylene produced can be determined in mL. 1M of gas=24 dm3 or 24,000 ml. Therefore, 1 mM of gas=24 ml. To calculate how many mM ethylene produced, divide amount by 24: mM ET=ml/24.

To calculate RATE: mM per hour per CFU, need to calculate mM as described above. Need to know how much of the headspace you sampled (if using calibration calculation e.g. 1 mL of headspace sampled has x mM gas but there is 6 mL headspace total so total ethylene produced=6x mM). If calculating using peak area % only the above step is not necessary-just need to know how much acetylene you added. Need to know the number of hours of incubation with Acetylene. Need to do TVCs to calculate CFU/ml then multiple your CFU value by the number of ml cultured e.g., 4 mL (gneg) or 30 mL (gpos).

Acetylene conversion to ethylene is presented under 0 mM and 5 mM conditions, as both raw numbers and normalized for cell growth at OD600. Results are shown in Table 3.

TABLE 3
Acetylene Reduction Assay for Genome Edited Strains
Rate = total ethylene mM/(time(h) × total CFU)

| | | 0 mM | | 5 mM |
|---|---|---|---|---|
| | 0 mM | normalized | 5 mM | normalized |
| Strain | Ethylene % | Ethylene % | Ethylene % | Ethylene % |
| PE77155-G123 | 3.92 | 19.77 | 2.53 | 7.73 |
| PE77155-G169 | 2.6 | 12.7 | 1.41 | 4.11 |
| PE77155-G168 | 12.73 | 57.33 | 14.3 | 40.01 |
| PE77155-G170 | 3.83 | 18.4 | 2.59 | 7.53 |
| PE77155-G171 | 5 | 25.45 | 6.37 | 25.01 |
| PE77155-G172 | 3.4 | 17.89 | 2.23 | 5.86 |
| PE77155-G122 | 4.31 | 21.03 | 2.35 | 0.03 |
| PE77155-G150 | 3.69 | 18.9 | 2.16 | 6.85 |
| PE77155-G151 | 12.01 | 59.77 | 17.53 | 50.23 |
| PE77155-G152 | 5.5 | 25.95 | 4.04 | 12.64 |
| PE77155-G153 | 1.9 | 9.2 | 2.91 | 9.42 |
| PE77155-G154 | 4.92 | 24 | 2.51 | 7.4 |
| PE77155-G121 | 6.86 | 33 | 0.01 | 0.04 |
| PE77155-G124 | 2.55 | 13.84 | 0.01 | 0.03 |
| PE77155-G127 | 23.81 | 105.82 | 0.84 | 2.54 |
| PE77155-G128 | 5.99 | 34.23 | 0.17 | 0.51 |
| PE77155-G129 | 3.15 | 11.4 | 3.21 | 7.68 |
| PE77155-G130 | 5.23 | 14.32 | 0.01 | 0.02 |
| PE77155-G182 | 6.84 | 32.48 | 0.17 | 0.42 |
| PE77155-G184 | 0.03 | 0.17 | 0.01 | 0.02 |
| PE77155-G183 | 14.55 | 84.6 | 0.46 | 1.47 |
| PE77155-G185 | 4.77 | 20.65 | 0.12 | 0.28 |
| PE77155-G186 | 11.56 | 53.95 | 7.38 | 25.41 |
| PE77155-G187 | 5 | 23.71 | 0.21 | 0.67 |
| PE77155-G155 | 9.19 | 54.04 | 0.13 | 0.39 |
| PE77155-G156 | 28.52 | 141.21 | 1.53 | 4.45 |
| PE77155-G157 | 0.13 | 0.9 | 0 | 0 |
| PE77155-G158 | 8.77 | 58.85 | 0 | 0 |
| PE77155-G159 | 1.24 | 6.85 | 2.05 | 7.2 |
| PE77155-G160 | 5.87 | 34.33 | 0.18 | 0.6 |
| PE77155-G188 | 17.44 | 72.22 | 0 | 0 |
| PE77155-G189 | 13.06 | 39.38 | 0 | 0 |
| PE77155-G190 | 13.64 | 47.12 | 0 | 0 |
| PE77155-G191 | 15.99 | 58.13 | 0 | 0 |
| PE77155-G192 | 13.48 | 69.13 | 0 | 0 |
| PE77155-G188 | 15.87 | 53.87 | 0 | 0 |
| PE77155-G193 | 13.04 | 39.14 | 0 | 0 |
| PE77155-G194 | 16.66 | 111.81 | 0 | 0 |
| PE77155-G195 | 13.74 | 75.49 | 0 | 0 |
| PE77155-G196 | 15.64 | 54.19 | 0 | 0 |
| PE77155-G188 | 9.77 | 53.13 | 0 | 0 |
| PE77155-G199 | 8.75 | 69.17 | 0 | 0 |
| PE77155-G200 | 8.55 | 51.63 | 0 | 0 |
| CE17899-G134 | 2.4 | 35.29 | 0 | 0 |
| CE17899-G135 | 1.67 | 26.43 | 0 | 0 |
| PE77155-G208 | 8.47 | 85.13 | 0 | 0 |
| PE77155-G209 | 8.41 | 86.7 | 0.25 | 0.41 |
| PE77155-G188 | 5.6 | 26.71 | 0 | 0 |
| PE77155-G194 | 5.09 | 26.72 | 0 | 0 |
| PE77155-G188 | 4.96 | 24.47 | 0.04 | 0.13 |
| PE77155-G202 | 26.61 | 111.83 | 1.02 | 2.22 |
| PE77155-G204 | 5.47 | 23.46 | 0.96 | 1.23 |
| PE77155-G205 | 8.34 | 38.79 | 8.47 | 27.51 |
| PE77155-G206 | 6.53 | 25.61 | 0.01 | 0.04 |

### Root Colonization

Bacterial strains are prepared with the GFP gene integrated into its genome, using techniques known in the art. Seeds are treated with the strain(s), and using sterile technique, inoculated seeds are dropped into phytagel tubes. Tubes are placed in appropriate grow rooms and cover for 5 days to allow germination. The root tissue is separated from the seed and shoot, using EtOH and flame sterilized tweezers and scalpels. The root tissue is cut to all be in the same focal plane and pressed at the same level on 0.8% water-agar in a square plate to image. The same is performed for shoot tissue. The plant tissue is imaged for bacterial colonization using fluorescence microscopy.

### Biofilm Assay Protocol

This protocol is based on literature: “Effects of an EPS Biosynthesis Gene Cluster of Paenibacillus polymyxa WLY78 on Biofilm Formation and Nitrogen Fixation under Aerobic Conditions” (Chen 2021). Materials: Sterile 3 mL glass tubes, ‘Biofilm Broth (BFB)’ media, 0.1% Crystal Violet (aqueous) Solution, 40% Acetic acid. 7 days gave best overall biofilm results; some isolates can give better results over 5 days and start to break down after this timepoint. Prepare using sterile technique.

The recipe for BFB includes: 5 g/L KH2PO4, 5 g/L K2HPO4, 0.86 g/L Mono sodium glutamate, 0.1 g/L yeast extract, 1 g/L NH4Cl pH 7. Filter sterilizing after autoclaved: 36 g/L glucose, 0.03 g/L MgSO4.7H2O, 0.02 g/L CaCl2.2H2O, 1 ml/L Trace element solution.

The method steps are:
1. Streak isolates from −80 C.
2. Make spread plates of each isolate.
3. Autoclave 3 mL glass tubes in tube rack (x3/isolate), use foil as a cover
4. Harvest spread plates and OD600 balance to ~0.3
5. Fill each tube with 1 mL BFB.
6. Inoculate with 10 uL/tube of spread plate harvest.
7. Place foil cover back over tubes and incubate at 30 C, stationary for 7 days.
8. After 7 days, start by removing the culture from tubes using long (1250 uL) pipette tips-collect culture in 2 mL snap cap tubes.
9. Add water to the culture to reach final volume of 1 mL-Take OD600 reading.
10. Wash glass tubes using RO water; fill approx. half-way, hold tube, sealing the top and shake to dislodge excess cellular material. Rinse several times.
11. Remove excess water using long pipette tips.
12. Add 1 mL/tube of 0.1% Crystal Violet solution and incubate for 10 minutes at room temperature.
13. Remove Crystal Violet solution by pipette into a waste container (e.g., 50 mL falcon tube) and dispose in the incineration waste bin.
14. Rinse glass tubes until water runs clear.
15. Dry glass tubes (usually overnight).
16. Add 1 mL 40% acetic acid solution to dissolve stained biofilm ring.
17. Take OD570 reading.
18. Normalize OD570 by OD600 (if appropriate).

### Example 9: In Planta Testing

The edited microbes described above are tested in at least one species of plant, in replicate.

Plants are associated with the wild-type and/or edited microbes described above, and tested in the greenhouse as well as in larger-scale field trials. Association may be accomplished by any one or more of the following: seed treatment, foliar treatment, in-furrow application, drench, side-dress.

In one example, multiple replicates of corn (maize; Zea mays) plants are treated with the microbes described herein and grown for at least 19 days (range 19-34 days). Data collected included biomass, leaf area, plant height, root area, shoot Nitrogen, greenness, NDVI (capturing how much more near infrared light is reflected compared to visible red; a measure of the state of plant health based on how the plant reflects light at certain frequencies), NPCI (normalized pigment chlorophyll ratio index), PSRI (plant senescence reflectance index), and CCI (chlorophyll content index), and compared to an untreated control.

It is claimed:

1. A modified bacterium, wherein the bacterium is of the Genus Paenibacillus and comprises an edit in one or more loci of its genome; wherein the edit is selected from the group consisting of:
(a) a duplicated or higher-order replication of one or more components of the nif cluster;
(b) a heterologous promoter inserted before nifH, anfH, and/or vnfH;
(c) the introduction of anf, vnf, nif1, nif2, nif4, hesA2, katA, sodA, and/or glnK;
(d) duplication or higher-order replication of anf and/or vnf gene(s);
(e) upregulation of anf and/or vnf gene(s);
(f) upregulation anf and/or vnf genes/gene clusters by using GlnR/Site I/II regulation control;
(g) insertion of orf1 into the nif cluster of a strain that does not already comprise said orf1;
(h) altered distance between the nif cluster and the native chromosomal Origin of Replication;
(i) provision of multiple copies of nifB;
(j) transfer of all or part of the nif gene cluster from a first strain to a second strain, wherein the second strain possesses an improved characteristic;
(k) modification of the nitrogenase enzyne, wherein the modification is in one or more of the polypeptides comprising the nitrogenase enzyme;
(l) introduction of a hesA2 gene;
(m) modification of the nifH cluster, for example: duplication of the nitrogen fixation gene cluster using a “distant” nitrogen fixation gene cluster or synthetic cluster in a low copy number plasmid-based test system; duplication of the nitrogen fixation gene cluster using Subgroup II cluster with and without orf1 using a plasmid-based test system; duplication of the nitrogen fixation gene cluster using a cluster originating from a different species; duplication of the nitrogen fixation gene cluster using a cluster originating from the same species; duplication of the nitrogen fixation gene cluster using anf and/or vnf cluster using a plasmid-based test system;
(n) replication of all or part of the nif cluster, for example on a plasmid or into the microbe's genome via chromosomal integration;
(o) introduction of a nifA gene from a near relative of Paenibacillus;
(p) upregulation of O2 scavenging activity;
(q) duplication or higher-order replication of the catalase gene;
(r) upregulation of the SOD enzyme; and/or
(s) any plurality or combination of the preceding;
wherein the Paenibacillus bacterium displays an improved phenotype as compared to a Paenibacillus bacterium not comprising said edit, wherein the improved phenotype is selected from the group consisting of: increased acetylene reduction capability, improved biofilm formation, increased turbidity in culture, greater nitrogen fixation tolerance to oxygen levels, and any plurality and/or combination of the preceding.

2. The modified bacterium of claim 1, wherein the edit is achieved by the introduction of replicative plasmid.

3. The modified bacterium of claim 1, wherein the edit is achieved by the modification of a genomic polynucleotide.

4. A synthetic composition comprising the modified bacterium of claim 1.

5. The synthetic composition of claim 4, further comprising a formulation component and/or an agricultural composition.

6. The synthetic composition of claim 4, wherein the Paenibacillus bacterium is present at a concentration of at least about 10\hat{ }2 CFU/mL in a liquid formulation, or at least about 10\hat{ }2 CFU/gram in a non-liquid formulation.

7. The synthetic composition of claim 4, further comprising at least one additional microbe.

8. The synthetic composition of claim 4, wherein the plant element is a seed.

9. The synthetic composition of claim 4, wherein the plant element is a seed that comprises a transgene.

10. The synthetic composition of claim 4, wherein the plant element is a leaf.

11. The synthetic composition of claim 4, wherein the plant element is a root.

12. The synthetic composition of claim 4, wherein the plant element is a whole plant.

13. The synthetic composition of claim 4, wherein the plant element is a plant reproductive element.

14. The synthetic composition of claim 4, wherein the formulation component is selected from the group consisting of: a compound that improves the stability of the microbe, a preservative, a carrier, a surfactant, an anticomplex agent, and any combination thereof.

15. The synthetic composition of claim 4, wherein the agricultural composition comprises a fungicide, a nematicide, a bactericide, an insecticide, an herbicide, a micronutrient, a macronutrient, Nitrogen, Phosphorous, Potassium, or any plurality and/or combination of the preceding.

16. A plurality of synthetic compositions of claim 4, wherein said synthetic compositions are substantially confined within an object selected from the group consisting of: a tube, a bottle, a jar, an ampule, a package, a vessel, a bag, a box, a bin, an envelope, a carton, a container, a silo, a shipping container, a truck bed, and a case.

17. The plurality of synthetic compositions of claim 16, wherein the synthetic compositions are at a temperature below zero degrees Celsius.

18. A plurality of synthetic compositions of claim 16, wherein the synthetic compositions are stable at or above room temperature.

19. The synthetic composition of claim 4, wherein the plant element is obtained from a monocot plant.

20. The synthetic composition of claim 19, wherein the monocot plant is a C3 monocot plant.

21. The synthetic composition of claim 19, wherein the monocot plant is a C4 monocot plant.

22. The synthetic composition of claim 19, wherein the monocot plant is a dicot plant.

23. The synthetic composition of claim 4, wherein the growth medium comprises soil.

24. A plurality of synthetic compositions of claim 4, wherein the plurality of synthetic compositions is placed in the soil in a regular pattern with substantially equal spacing between each of the synthetic compositions.

25. A method of improving the health, yield, and/or vigor of a plant, the method comprising:
a. associating an element of the plant with the modified bacterium of claim 1;
b. growing a plant from the element of the crop plant in a medium that supports plant growth; and
c. assessing one or more characteristics of the plant, wherein at least one of said characteristics is improved, as compared to the same characteristic of a plant not obtained from an element associated with the Paenibacillus bacterium of (a).

26. The method of claim 25, wherein the one or more characteristics of (d) includes an improvement of nitrogen fixation, increase in biomass, increase in leaf area, increase in plant height, increase in root area, increase in shoot nitrogen composition, increase in greenness, increase in NDVI, increase in NPCI, increase in PSRI, increase in CCI, increase in yield, and any combination of the preceding.

27. The method of claim 25, further comprising at least one additional microbe.

28. The method of claim 25, wherein the associating an element of the crop plant with the modified bacterium comprising an edit in one or more loci of its genome is accomplished by a method selected from the group consisting of: in-furrow application, soil drench application, side-dress application, foliar application, pre-harvest fruit application, and any combination of the preceding.

29. The method of claim 25, wherein the associating an element of the crop plant with the modified bacterium comprising an edit in one or more loci of its genome is accomplished by coating said plant element with a liquid formulation of the bacterium.

30. The method of claim 25, wherein the associating an element of the crop plant with the modified bacterium comprising an edit in one or more loci of its genome is accomplished by coating said plant element with a substantially non-liquid formulation of the bacterium.

31. The method of claim 25, wherein said plant element is a seed.

32. The method of claim 25, wherein said plant element is a leaf.

33. The method of claim 25, wherein said plant element is a root.

34. The method of claim 25, wherein said plant element is a whole plant.

35. The modified bacterium of claim 1, wherein the bacterium displays an improved phenotype as compared to an unmodified bacterium, wherein the improved phenotype is selected from the group consisting of: increased acetylene reduction capability, improved biofilm formation, increased turbidity in culture, greater nitrogen fixation tolerance to oxygen levels, and any combination of the preceding.

36. A substantially pure composition comprising the modified bacterium of claim 1.

37. A bacterial culture comprising the modified bacterium of claim 1.

38. A fermentation culture comprising the modified bacterium of claim 1.

39. An agricultural composition, comprising the modified bacterium of claim 1 and an agriculturally-acceptable carrier.

40. The agricultural composition of claim 37, further comprising a plant or plant element, wherein the modified bacterium is present in the agricultural composition in an amount effective for producing an improved phenotype in the plant.

41. The agricultural composition of claim 37, wherein the improved phenotype is an increase in the health, yield, and/or vigor of the plant.

42. A method for improving the nitrogen fixation capability of a Paenibacillus bacterium, the method comprising:
(a) the nifHDK cluster in a Subgroup I strain is duplicated into that same strain (e.g., nifBHDK-nifBHDK-nifBHDK);
(b) anf and/or vnf may be introduced into Paenibacillus, such as Subgroup I strains (e.g., nifBHDK-anfHDGK-vnfHDGK);
(c) gene expression across nifH, anfH, and/or vnfH may be synchronized in a strain with the same promoter and nifB gene;
(d) the nifB genes in a Subgroup II strain are streamlined;
(e) different promoters and/or other regulatory elements may be changed, modified, or swapped;
(f) anf and/or vnf gene(s) may be introduced into a strain of Subgroup I, such as Paenibacillus polymyxa; The anf/vnf cluster is less sensitive to O2, and introducing into polymyxa may make it less sensitive to oxygen, or function in the absence or limitation of a rare element;
(g) anf and/or vnf gene(s) may be duplicated or higher-order replicated;
(h) anf and/or vnf gene(s) may be upregulated;
(i) orf1 may be inserted into the nif cluster of a Subgroup I strain;
(j) the distance between the cluster and the ORI may be altered, to obtain more consistent expression;
(k) duplication of some or all of the cluster within the microbe's genome may be effected, for example by providing multiple copies of nifB, as the instigator of the cluster; The nifB gene of P. polymyxa is longer than other nifBs;
(l) all or part of the nif gene cluster may be transferred from one strain to another, which possesses an improved characteristic (such as colonization of plant tissue);
(m) the nitrogenase protein may be modified to a hybrid of domains of cofactor-utilizing enzymes, to create a novel protein that has more promiscuous activity and not as dependent upon a particular environment as it would be capable of utilizing a variety of cofactors;
(n) the hesA2 gene may be introduced into P. polymyxa, to enhance the promiscuity of the nitrogenase enzyme; a hesA2 gene may be introduced into a Paenibacillus strain to enhance nitrogen fixation activity;
(o) the nitrogen fixation gene cluster is modified, for example: duplication of the nitrogen fixation gene cluster using “distant” nitrogen fixation gene cluster or synthetic cluster in a low copy number plasmid-based test system; duplication of the nitrogen fixation gene cluster using Subgroup II cluster with and without orf1 using a plasmid-based test system; duplication of the nitrogen fixation gene cluster using anf cluster using a plasmid-based test system
(p) all or part of the nif cluster is replicated, for example on a plasmid or into the microbe's genome via chromosomal integration;
(q) an artificial dimer of nifH may be created using non-naturally occurring combinations of subunits;
(r) a nifA gene from a near relative of Paenibacillus (e.g., Frankia) is introduced into the Paenibacillus bacterium as a binder for a negative regulator;
(s) the scavenging of O2 activity is upregulated;
(t) the catalase gene is duplicated (or triplicated, or more);
(u) a katA gene is inserted;
(v) a glnK gene is inserted;
(w) a hesA2 gene is inserted;
(x) a sodA gene is inserted;
(y) the SOD enzyme is upregulated; and/or
(z) any plurality or combination of the preceding.
